(19) United States
(12) Patent Application Publication
(10) Pub. No.: US 2010/0197018 A1
(43) Pub. Date: Aug. 5, 2010

(54) USE OF SUBSTRATES AS PHARMACOLOGICAL CHAPERONES

(75) Inventor: Benjamin Mugrage, Cranbury, NJ (US)

Correspondence Address: HOFFMANN & BARON, LLP, 6900 JERICHO TURNPIKE, SYOSSET, NY, 11791, US

(73) Assignee: AMICUS THERAPEUTICS, INC., Cranbury, NJ (US)

(21) Appl. No.: 12/595,081

(22) PCT Filed: Apr. 11, 2008
(86) PCT No.: PCT/US08/60094

§ 371 Date: Mar. 19, 2010

Related U.S. Application Data
(60) Provisional application No. 60/911,710, filed on Apr. 13, 2007.

(51) Int. Cl. C12N 5/071

(52) U.S. Cl. 435/375

(57) ABSTRACT

Provided is a method of enhancing the activity of lysosomal enzymes using substrates that are derivatives of natural substrates as pharmacological chaperones.

### CROSS-REFERENCE TO RELATED APPLICATIONS

This application claims priority to U.S. Provisional Application Ser. No. 60/911,710 filed Apr. 13, 2007, which is hereby incorporated by reference in its entirety herein.

### FIELD OF THE INVENTION

The present invention relates to a method for treating lysosomal storage diseases using pharmacological chaperones which are substrates or substrate analogs for the enzyme which is deficient in the lysosomal storage disease due to a conformational mutation. This method also can be applied to diseases associated with other enzyme deficiencies due to conformational mutations of the associated enzyme.

### BACKGROUND

Proteins are synthesized in the cytoplasm, and the newly synthesized proteins are secreted into the lumen of the endoplasmic reticulum (ER) in a largely unfolded state. In general, protein folding is governed by the principle of self assembly. Newly synthesized polypeptides fold into their native conformation based on their amino acid sequences (Anfinsen et al., Adv. Protein Chem. 1975; 29:205-300). In vivo, protein folding is complicated, because the combination of ambient temperature and high protein concentration stimulates the process of aggregation, in which amino acids normally buried in the hydrophobic core interact with their neighbors non-specifically. To avoid this problem, protein folding is usually facilitated by a special group of proteins called chaperones, which prevent nascent polypeptide chains from aggregating by binding to unfolded protein such that the protein refolds in the native conformation (Hartl, Nature 1996; 381:571-580).

Endogenous molecular chaperones are present in virtually all types of cells and in most cellular compartments. Some are involved in the transport of proteins and permit cells to survive under stresses such as heat shock and glucose starvation (Gething et al., Nature 1992; 355:33-45; Caplan, Trends Cell. Biol. 1999; 9:262-268; Lin et al., Mol. Biol. Cell. 1993; 4:109-1119; Bergeron et al., Trends Biochem. Sci. 1994; 19:124-128). Among the endogenous chaperones, BiP (immunoglobulin heavy-chain binding protein, Grp78) is the best characterized chaperone of the ER (Haas, Curr. Top. Microbiol. Immunol. 1991; 167:71-82). Like other chaperones, BiP interacts with many secretory and membrane proteins within the ER throughout their maturation. When nascent protein folding proceeds smoothly, this interaction is normally weak and short-lived. Once the native protein conformation is achieved, the molecular chaperone no longer interacts with the protein. BiP binding to a protein that fails to fold, assemble, or be properly glycosylated becomes stable, and usually leads to degradation of the protein through the ER-associated degradation pathway. This process serves as a “quality control” system in the ER, ensuring that only those properly folded and assembled proteins are transported out of the ER for further maturation, and improperly folded proteins are retained for subsequent degradation (Hurtley et al., Annu. Rev. Cell. Biol. 1989; 5:277-307). Due to the combined actions of the inefficiency of the thermodynamic protein folding process and the ER quality control system, only a fraction of nascent (non-mutated) proteins become folded into a functional conformation and successfully exit the ER.

### Pharmacological Chaperones Derived From Specific Enzyme Inhibitors Rescue Mutant Enzymes and Enhance Wild-Type Enzymes

It has previously been shown that small molecule inhibitors of enzymes associated with lysosomal storage disorders (LSDs) can both rescue folding and activity of the mutant enzyme, and enhance folding and activity of the wild-type enzyme (see U.S. Pat. Nos. 6,274,597; 6,583,158; 6,589,964; 6,599,919; and 6,916,829, all incorporated herein by reference). In particular, it was discovered that administration of small molecule derivatives of glucose and galactose, which were reversible specific competitive inhibitors of mutant enzymes associated with LSDs, effectively increased in vitro and in vivo stability of the mutant enzymes and enhanced the mutant enzyme activity. The original theory behind this strategy is as follows: since the mutant enzyme protein folds improperly in the ER (Ishii et al., Biochem. Biophys. Res. Comm. 1996; 220: 812-815), the enzyme protein is retarded in the normal transport pathway (ER→Golgi apparatus→endosome→lysosome) and rapidly degraded. Therefore, a compound which stabilizes the correct folding of a mutant protein will serve as an active site-specific chaperone for the mutant protein to promote its smooth escape from the ER quality control system. This strategy was demonstrated initially using galactose as the chaperone for mutant α-galactosidase A (α-Gal-A; Okuyima et al., Biochem Biophis Res Comm. 1995; 214: 1219-24). However, galactose is a product of α-Gal-A activity and not a true inhibitor (or substrate). Further, large doses were required to restore mutant α-Gal-A activity in the only patient to whom it was administered, making it an impractical therapeutic candidate. Enzyme inhibitors also occupy the catalytic center, resulting in stabilization of enzyme conformation in cells in culture and in animals. However, since they are reversible and can dissociate from the enzyme once it is out of the ER, they do not prevent subsequent binding of the substrate and thus, do not inhibit the enzyme's function. These specific pharmacological chaperones were designated “active site-specific chaperones (ASSCs)” since they bound (reversibly) in the active site of the enzyme.

This strategy was applied beyond lysosomal storage diseases to other diseases associated with conformational mutants, and also to diseases or conditions not associated with conformational mutants but where increased activity of the wild-type enzyme would be beneficial. Examples of other conformational diseases include cancers associated with mutant PTEN, Alzheimer's disease associated with mutant α-secretase, and Parkinson's disease associated with heterozygous mutations in glucocerebrosidase. See co-owned U.S. provisional application Ser. Nos. 60/799,969, filed May 12, 2006; 60/800,071, filed May 12, 2006, and U.S. patent application Ser. No. 11/449,528, filed on Jun. 8, 2006. Increasing the activity of the non-mutant, wild-type enzymes in patients at risk of developing these conditions may prevent, delay the development of, or mitigate the severity of these diseases.

In addition to rescuing the mutant enzymes, the ASSCs can enhance ER secretion and activity of recombinant wild-type enzymes. An ASSC facilitates folding of overexpressed wild-type enzyme, which is otherwise retarded in the ER quality control system because overexpression and over production of the enzyme exceeds the capacity of the ER and leads to protein aggregation and degradation. Thus, a compound that induces a stable molecular conformation of an enzyme during folding serves as a “chaperone” to stabilize the enzyme in a proper conformation for exit from the ER. As noted above, for enzymes, such compounds unexpectedly turned out to be specific competitive inhibitors of the enzyme.

However, although there are known competitive inhibitors for many lysosomal and other enzymes, there are no known inhibitors (or other small molecule compounds) which specifically and reversibly bind to other enzymes which are associated with disease states. Thus, the present invention provides a method for enhancing the activity of enzymes, in particular, for lysosomal enzymes for which the only known agents that specifically bind to the enzymes are the substrates or substrate analogs. U.S. published patent application 2005/015934 to Schuchman describes sphingomyelin and ceramide analogs as potential chaperones to rescue mutant acid sphingomyelinase associated with Niemann-Pick Type A and B. Ceramide is a product of the hydrolysis of sphingomyelin to ceramide and phosphocholine. Two of the sphingomyelin analogs described therein may be substrate analogs although it is unclear whether those analogs would be hydrolyzed by acid spingomyelinase similar to the natural substrate. However, they can bind to and inhibit sphingomyelin in vitro.

### SUMMARY OF THE INVENTION

The present invention provides a method of increasing the activity of a lysosomal enzyme in a cell by contacting the cell with a substrate or substrate analog specific for the enzyme, with the proviso that the lysosomal enzyme is not acid sphingomyelinase.

In a specific embodiment, the lysosomal enzyme is deficient due to a conformational mutation.

In another specific embodiment, the lysosomal enzyme is wild-type. In one aspect of the invention, the lysosomal enzyme is iduronate-2-sulfatase and the substrate or analog is heparan sulfate; dermatan sulfate; O-(α-L-idopyranosyluronic acid 2-sulfate)-(1-4)-(2,5-anhydro-D-mannitol-1-t 6-sulfate (IdA-Ms); L-O-(α-iduronic acid 2-sulphate-(1-4)-D-β-2,5-anhydro-mannitol (IdoA2S-anM); L-O-(α-iduronic acid 2-sulphate)-(1-4)-D-β-2,5-anhydro-mannitol 6-sulphate (IdoA2S-anM6S); O-(α-L-idopyranosyluronic acid)-(1-3)-2,5-anhydro-D-talitol 4-sulfate (IdoA-anT4S); O-(α-L-idopyranosyluronic acid 2-sulfate)-(1-3)-2,5-anhydro-D-talitol 4-sulfate (IdoA2S-anT4S); L-O-(α-iduronic acid 2 sulphate)-D-β-α-glucosamine 6-sulphate)-(1-4)-L-O-(α-iduronic acid 2-sulphate)-(1-4)-D-O-anhydro-mannitol 6-sulphate (IdoA2S-GlcNH6S-IdoA2S-anM6S); L-O-(α-iduronic acid 2 sulphate)-(1-4)-D-β-(α-2-sulphaminoglucosamine)-(1-4)-O-(β-D-glucuronic or α-L-iduronic acid)-(1-4) D-O(α-N-acetylglucosamine-(1-3)-D-O-]-gulonic acid (IdoA2S-GlcNS-UA-GlcNAc-GlcOA); O-(β-D-glucopyranosyluronic acid)-(1-3)-2,5-anhydro-D-talitol 4-sulfate (GlcA-anT4S); O-(β-D-glucopyranosyluronic acid)-(1-3)-2,5-anhydro-D-talitol 6-sulfate (GlcA-anT6S); and O-(α-L-idopyranosyluronic acid)-(1-3)-2,5-anhydro-D-talitol (IdoA-anT); or O-(α-L-idopyranosyluronic acid-2-sulphate)-(1-4)-2,5-anhydro-D-mannitol-6-sulphate.

In a second aspect of the invention, the lysosomal enzyme is 1-leparan-N-sulfatase and the substrate or substrate analog is heparan; heparin; O-α-2-sulphaminoglucosamine)-(1-4) O-L-(α-iduronic-acid 2-sulphate)-(1-4)-O-D-(2,5)-anhydro-mannitol 6-sulphate (GlcNS-IdoA2S-anM6S); O-(α-2-sulphaminoglucosamine)-(1-4)-L-O-(α-iduronic acid)-(1-4)-O-D-(α-2-sulphaminoglucosamine)-(1-3)-L-idonic acid (GlcNS-IdoA-GlcNS-IdOA); O-(α-2-sulphaminoglucosamine)-(1-4)-O-L-iduronic acid (GlcNS-IdOA); O-(α-2-sulphaminoglucosamine 6-sulphate)-(1-3)-L-idonic acid (GlcN6S-IdOA); O-(α-2-sulphaminoglucosamine 6 sulphate)-(1-3)-L-idonic acid (GlcNS6S-IdOA); O-(α-2-sulphaminoglucosamine)-(1-4)-L-idose) (GlcNS-Ido); O-(α-2-sulphaminoglucosamine 6-sulphate)-(1-4)-L-idose 2-sulphate (GlcNS6S-Ido2S); O-(α-2-sulphaminoglucosamine 6-sulphate)-(1-4)-L-idose (GlcNS6S-Ido); O-(α-2-sulphaminoglucosamine)-(1-4)-L-6-idose 2-sulphate (GlcNS-Ido2S); 2-sulphoamino-glucosamine (GlcNS); or 2-sulphoamino-galactosamine (GalNS).

In a third aspect of the invention, the lysosomal enzyme is α-glucosaminide N-acetyltransferase and the substrate or substrate analog is heparan sulfate; α-N-acetylglucosamine; or 0-(2-amino-2-deoxy-α-D-glucopyranosyl N-sulphate)-(1-4)-β-D-uronicacid-(1-4)-(2-amino-2-deoxy-α-D-glucopyranosyl N-sulphate)-(1-3)-L-idonic acid (or -2,5-anhydro-L-idonic acid or -L-gulonic acid).

In a fourth aspect of the invention, the lysosomal enzyme is N-acetyl-glucosamine-6-sulfate sulfatase and the substrate or substrate analog is heparan sulfate; keratan sulfate; N-acetyl-glucosamine 6-sulfate; glucose 6-sulfate; 0-α-D-6-sulfo-2-acetamido-2-deoxyglucosyl-(1-4)-O-uronosyl-(1-4)-2,5-anhydro-D-mannitol (GlcNAc(6S)UA-aMan-ol); O-(α-L-iduronic acid 2-sulphate)-(1-4)-D-β-(α-2-sulphaminoglucosamine 6 sulphate)-(1-4)-L-O-(α-iduronic acid 2-sulphate)-(1-4)-D-O-2,5-anhydro[1-³H]mannitol 6-sulphate (IdoA2S-GlcNS6S-IdoA2S-anM6S); O-(α-N-acetylglucosamine 6-sulphate)-(1-4)-L-O-(α-iduronic acid 2-sulphate)-(1-4)-D-O-2,5-anhydro-mannitol 6 sulphate(GluNAc6S-IdoA2S-anM6S); O-α-glucosamine 6-sulphate)-(1-4)-L-O-(α-iduronic acid 2-sulphate)-(1-4)-D-β-2,5-anhydro-mannitol 6-sulphate (GlcNH6S-IdoA2S-anM6S); or O-(α-N-acetylglucosamine 6-sulphate)-(1-3)-L-idonic acid (GlcNAc6S-IdOA).

In a fifth aspect of the invention, the lysosomal enzyme is N-acetyl-galactosamine-6-sulfate-sulfatase and the substrate or substrate analog is keratan sulfate; chondroitin-6-sulfate; hyaluronidase-degraded C-6-S tetrasaccharide; 6-sulfo-N-acetylgalactosamine-glucuronic acid-6-sulfo-N-acetyl-1-galactosaminitol; or N-acetylgalactosamine 6-sulfate-((β, 1-4)-glucuronic acid-(β, 1-3 (-N-acetylgalactosaminitol 6-sulfate)).

In a sixth aspect of the present invention, the lysosomal enzyme is Arylsulfatase A and the substrate or substrate analog is cerebroside sulfate; 4-nitrocatechol sulfate; dehydroepiandrosterone sulfate; cerebroside-3-sulfate; ascorbate-2-sulfate; sodium 2-hydroxy-5-nitrobenzylsulfonate monohydrate (Na(+)×C(7)H(6)NO(6)S(−)×H(2)O); N-[7-Nitrobenz-2-oxa-1,3-diazol-4-yl]psychosine sulfate (NBD-PS); 2-(1-pyrene)dodecanoyl cerebroside sulfate (P12-sulfatide); or 12(1-pyrenesulfonylamido)dodecanoyl cerebroside sulfate (PSA12-sulfatide).

In a seventh aspect of the invention, the lysosomal enzyme is Arylsulfatase B and the substrate or substrate analog is iduronate sulfate; dermatan sulfate; chondroitin sulfate; p-nitrocatechol sulfate; GalNAc4S-GlcA-GalitoINAc4S; chondroitin 4-sulfate-tetrasaccharide; or N-acetygalactosamine 4-sulfate-(1-4)-beta-glucuronic acid-(1-3)-beta-N-acetylgalactosaminitol 4-sulfate.

In an eight aspect of the invention, the lysosomal enzyme is acid ceramidase and the substrate or substrate analog is ceramide; N-stearoylsphingosine; N-stearoyldihydro-sphingosine; N-oleosphingosine; or N-lauroylsphingosine.

In a ninth aspect of the invention, the lysosomal enzyme is N-Acetylglucosamine-1-Phosphotransferase and the substrate or substrate analog is UDP-N-acetylglucosamine or α-methyl-mannoside.

### BRIEF DESCRIPTION OF THE DRAWINGS

FIG. 1. FIG. 1 provides a cartoon depicting a potential mechanism for using substrates as chaperones.

FIG. 2A-B. FIG. 2A depicts the structure of heparan sulfate. FIG. 2B depicts the structure of heparan sulfate analog GlcNS6S-IdOA.

### DETAILED DESCRIPTION

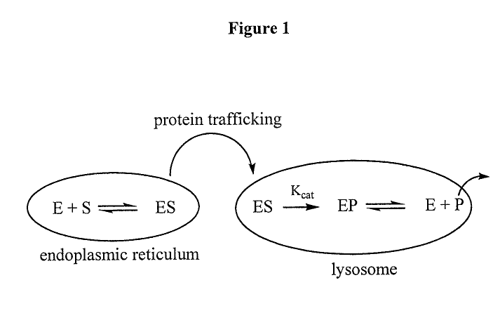

Provided is a method for increasing the activity of enzymes using substrates or substrate analogs or derivatives for the enzymes. The substrate binds to a target enzyme in the endoplasmic reticulum (ER) and stabilizes the enzyme in a conformation that permits it to exit the ER and traffick to its native location in the cell, such as the lysosome. Once out of the ER, the bound substrate or analog or derivative is processed by the enzyme, the product dissociates from the enzyme, and the enzyme is available to process other substrates. The method contemplates use for both wild-type enzyme and enzymes which are conformational mutants. This method is especially suited for substrates which have a strong affinity for the enzyme in the ER, which favors the formation of an enzyme-substrate complex (ES), but has a low turnover rate (low K_{cat}, low Kₘ), which enables the substrate to remain bound for a sufficient period to chaperone the enzyme from the ER, such as to its native cellular location (FIG. 1).

### DEFINITIONS

The terms used in this specification generally have their ordinary meanings in the art, within the context of this invention and in the specific context where each term is used. Certain terms are discussed below, or elsewhere in the specification, to provide additional guidance to the practitioner in describing the compositions and methods of the invention and how to make and use them.

As used herein, the term “pharmacological chaperone,” or sometimes “specific pharmacological chaperone” (“SPC”), refers to a molecule that specifically binds to a protein, particularly an enzyme, and has one or more of the following effects: (i) enhancing the formation of a stable molecular conformation of the protein; (ii) enhances proper trafficking of the protein from the ER to another cellular location, preferably a native cellular location, i.e., preventing ER-associated degradation of the protein; (iii) preventing aggregation of conformationally unstable, i.e., misfolded proteins; (iv) restoring or enhancing at least partial wild-type function, stability, and/or activity of the protein; and/or (v) improving the phenotype or function of the cell harboring a mutant protein. Thus, a pharmacological chaperone is a molecule that specifically binds to a protein, resulting in proper folding, trafficking, non-aggregation, and/or activity of that protein. In the context of the present invention, the specific pharmacological chaperones are substrates, or substrate analogs or derivatives, of the enzymes.

As used herein, the term “pharmacological chaperone” does not refer to endogenous chaperones, such as BiP, or to non-specific agents which have demonstrated non-specific chaperone activity against various proteins, such as glycerol, DMSO or deuterated water, i.e., chemical chaperones (see Welch et al., Cell Stress and Chaperones 1996; 1(2):109-115; Welch et al., Journal of Bioenergetics and Biomembranes 1997; 29(5):491-502; U.S. Pat. No. 5,900,360; U.S. Pat. No. 6,270,954; and U.S. Pat. No. 6,541,195).

As used herein, the term “substrate” refers to a molecule that is acted upon (i.e., modified) by an enzyme. According to the present invention, this term refers to an enzyme's natural or physiological substrate that is unmodified by human intervention. Examples of natural substrates for some lysosomal enzymes can be found in Tables 2.

As used herein, the terms “substrate analog” or “substrate derivative” refer to substrates which are modified from their natural or endogenous physiological state, either by nature or by human intervention, and which retain capability to be modified by the enzyme which modifies the corresponding natural or endogenous physiological substrate. More particularly, a “substrate analog” or “substrate derivative” refers to synthetic (artificial) or natural chemical compounds which resemble endogenous physiological enzyme substrates in structure and/or function. Typically substrate analogs and derivatives exhibit different physical properties than the natural or physiological substrate, including binding affinities (Km), and/or turnover rate (Kcat). Substrate analogs or derivatives often are smaller than the natural or physiological substrate. According to the present invention, substrate analogs or derivatives used as substrates may contain a detectable label, such as with a fluorogenic, chromogenic, or other type of label. One specific example of a fluorescent label is 4-methylumbelliferone (4-MU).

As used herein, the term “specifically binds” refers to the interaction of a pharmacological chaperone, i.e., substrate or substrate analog or derivative, with a particular protein, specifically, an interaction with amino acid residues of the protein that directly participate in contacting the pharmacological chaperone. A pharmacological chaperone specifically binds a target protein, e.g., lysosomal enzyme, to exert a chaperone effect on that enzyme and not a generic group of related or unrelated enzymes. In the case of an enzyme protein, the amino acid residues of the enzyme that interact with the chaperone are typically at the “active site” of the enzyme.

The “active site” for enzyme proteins is defined as the region of the enzyme which binds a substrate and catalyzes the reaction with or modification of the substrate.

The term “Vmax” refers to the maximum initial velocity of an enzyme catalyzed reaction, i.e., at saturating substrate levels. The term “Km” is the substrate concentration required to achieve one-half Vmax. The Kcat is defined as the Vmax divided by the total enzyme concentration, i.e., the maximum number of molecules of substrate which can be converted into product per enzyme molecule per unit time (the turnover number).

As used herein, the terms “enhance conformational stability” or “increase conformational stability” refer to increasing the amount or proportion of a protein that adopts a functional conformation in a cell contacted with a pharmacological chaperone, e.g., substrate, that is specific for the protein, relative to a protein in a cell (preferably of the same cell-type or the same cell, e.g., at an earlier time) not contacted with the pharmacological chaperone specific for the protein. In one embodiment, the cells do not express a conformation mutant. In another embodiment, the cells do express a mutant polynucleotide encoding a polypeptide e.g., a conformational mutant protein.

As used herein, the terms “enhance activity” or “increase activity” refer to increasing the activity of a protein, as described herein, in a cell contacted with a pharmacological chaperone specific for the protein, relative to the activity of the protein in a cell (preferably of the same cell-type or the same cell, e.g., at an earlier time) not contacted with the pharmacological chaperone specific for the protein. This term also refers to enhancing protein trafficking and enhancing protein expression level as defined directly below.

As used herein, the terms “enhance protein trafficking” or “increase protein trafficking” refer to increasing the efficiency of transport of a protein from the ER to another location in a cell contacted with a pharmacological chaperone specific for the protein, relative to the efficiency of transport of the protein in a cell (preferably of the same cell-type or the same cell, e.g., at an earlier time) not contacted with the pharmacological chaperone specific for the protein.

As used herein, the terms “enhance protein level” or “increase protein level” refer to increasing the level of a target protein in a cell contacted with a pharmacological chaperone specific for the protein, relative to the level of the protein in a cell (preferably of the same cell-type or the same cell, e.g., at an earlier time) not contacted with the pharmacological chaperone specific for the protein.

The term “stabilize a proper conformation” refers to the ability of a pharmacological chaperone, e.g., substrate or substrate analog or derivative, to induce or stabilize a conformation of a mutated target protein that is functionally equivalent to the conformation of the corresponding wild-type protein. The term “functionally equivalent” means that while there may be minor variations in the conformation (almost all proteins exhibit some conformational flexibility in their physiological state), this conformational flexibility does not result in (1) protein aggregation, (2) elimination through the endoplasmic reticulum-associated degradation pathway, (3) impairment of protein function, e.g., loss of activity, and/or (4) improper transport within the cell, e.g., localization to the lysosome, to significantly lesser degree than that of the wild-type protein.

The term “stable molecular conformation” refers to a conformation of a protein, i.e., a lysosomal enzyme, induced by a pharmacological chaperone, that provides at least partial wild-type function in the cell. For example, a stable molecular conformation of a mutant lysosomal enzyme would be one where the enzyme escapes from the ER and traffics to the lysosome as for a wild-type, instead of misfolding and being degraded. In addition, a stable molecular conformation of a mutated protein may also possess full or partial protein activity, e.g., lysosomal hydrolase activity. However, it is not necessary that the stable molecular conformation have all of the functional attributes of the wild-type protein.

The term “protein activity” refers to the normal physiological function of a wild-type protein in a cell. For example, the activity of a lysosomal enzyme (lysosomal enzyme activity) can include hydrolysis of a substrates including cellular lipids and carbohydrates. Such functionality can be tested by any means known to establish functionality of such a protein. For example, assays using fluorescent artificial substrates can be used to determine hydrolytic activity. Such assays are well known in the art. See e.g., Hopwood, J. Biol. Chem. 1999; 274: 37193-99 describes the production of recombinant sulfamidase. In addition, Braulke et al., Hum Mutation. 2004; 23:559-66, describes a means to assess transport, enzymatic activity and stability of mutant sulfamidase enzymes in cellular environments. In addition, a murine model for missense mutations in lysosomal sulfamidase has been described by Hopwood, Glycobiology. 2001; 11: 99-103. A spontaneous murine model for Sanfilippo Type IIIa is described in Bhattacharyya et al., Glycobiology. 2001; 11: 99-103.

The term “wild-type enzyme” refers to enzymes encoded by polypeptides that have the ability to achieve a functional conformation in the ER, achieve proper localization within the cell, and exhibit wild-type activity (e.g., lysosomal hydrolase activity). This term includes polypeptides, such as orthologs and homologs and allelic variants, which may differ from each other but whose encoded enzyme product exhibits the aforementioned wild-type activity.

A “lysosomal enzyme” refers to any enzyme that functions in the lysosome. Lysosomal enzymes include, but are not limited to, those listed in Tables 1 and 2. Additional lysosomal enzymes include, but are no limited, to α-glucosidase, acid β-glucosidase (glucocerebrosidase), α-galactosidase A, acid β-galactosidase, galactocerebrosidase, acid α-mannosidase, acid β-mannosidase, α-L-fucosidase, α-N-acetylglucosam in idase, α-N-acetylgalactosaminidase, β-hexosaminidase A, β-hexosaminidase B, α-L-iduronidase, β-glucuronidase, sialidase and acid sphingomyelinase

Certain tests may evaluate attributes of a protein that may or may not correspond to its actual in vivo function, but nevertheless are aggregate surrogates of protein functionality, and wild-type behavior in such tests is an acceptable consequence of the protein folding rescue or enhancement techniques of the present invention. One such activity in accordance with the invention is appropriate transport of a lysosomal enzyme from the endoplasmic reticulum to the lysosome.

As used herein the term “mutant protein” refers to a polypeptide translated from a gene containing a genetic mutation that results in an altered amino acid sequence. In one embodiment, the mutation results in a protein that does not achieve a native conformation under the conditions normally present in the ER, when compared with wild-type protein, or exhibits decreased stability or activity as compared with wild-type protein. This type of mutation is referred to herein as a “conformational mutation,” and the protein bearing such a mutation is referred as a “conformational mutant.” The failure to achieve this conformation results in protein being degraded or aggregated, rather than being transported through a normal pathway in the protein transport system to its native location in the cell or into the extracellular environment. In some embodiments, a mutation may occur in a non-coding part of the gene encoding a protein that results in less efficient expression of the protein, e.g., a mutation that affects transcription efficiency, splicing efficiency, mRNA stability, and the like. By enhancing the level of expression of wild-type as well as conformational mutant variants of the protein, administration of a pharmacological chaperone can ameliorate a deficit resulting from such inefficient protein expression.

The terms “therapeutically effective dose” and “effective amount” refer to the amount of the specific pharmacological chaperone that is sufficient to result in a therapeutic response. A therapeutic response may be any response that a user (e.g., a clinician) will recognize as an effective response to the therapy, including the foregoing symptoms and surrogate clinical markers. Thus, a therapeutic response will generally be an amelioration of one or more symptoms of a disease or disorder, e.g., a lysosomal storage disease, such as those known in the art for the disease or disorder, e.g., neurological symptoms.

The phrase “pharmaceutically acceptable” refers to molecular entities and compositions that are physiologically tolerable and do not typically produce untoward reactions when administered to a human. Preferably, as used herein, the term “pharmaceutically acceptable” means approved by a regulatory agency of the federal or a state government or listed in the U.S. Pharmacopeia or other generally recognized pharmacopeia for use in animals, and more particularly in humans. The term “carrier” refers to a diluent, adjuvant, excipient, or vehicle with which the compound is administered. Such pharmaceutical carriers can be sterile liquids, such as water and oils. Water or aqueous saline solutions and aqueous dextrose and glycerol solutions are preferably employed as carriers, particularly for injectable solutions. Suitable pharmaceutical carriers are described in “Remington's Pharmaceutical Sciences” by E.W. Martin, 18th Edition, or other editions.

The terms “about” and “approximately” shall generally mean an acceptable degree of error for the quantity measured given the nature or precision of the measurements. Typical, exemplary degrees of error are within 20 percent (%), preferably within 10%, and more preferably within 5% of a given value or range of values. Alternatively, and particularly in biological systems, the terms “about” and “approximately” may mean values that are within an order of magnitude, preferably within 5-fold and more preferably within 2-fold of a given value. Numerical quantities given herein are approximate unless stated otherwise, meaning that the term “about” or “approximately” can be inferred when not expressly stated.

As used herein, the term “isolated” means that the referenced material is removed from the environment in which it is normally found. Thus, an isolated biological material can be free of cellular components, i.e., components of the cells in which the material is found or produced. In the case of nucleic acid molecules, an isolated nucleic acid includes a PCR product, an mRNA band on a gel, a cDNA, or a restriction fragment. In another embodiment, an isolated nucleic acid is preferably excised from the chromosome in which it may be found, and more preferably is no longer joined to non-regulatory, non-coding regions, or to other genes, located upstream or downstream of the gene contained by the isolated nucleic acid molecule when found in the chromosome. In yet another embodiment, the isolated nucleic acid lacks one or more introns. isolated nucleic acids include sequences inserted into plasmids, cosmids, artificial chromosomes, and the like. Thus, in a specific embodiment, a recombinant nucleic acid is an isolated nucleic acid. An isolated protein may be associated with other proteins or nucleic acids, or both, with which it associates in the cell, or with cellular membranes if it is a membrane-associated protein. An isolated organelle, cell, or tissue is removed from the anatomical site in which it is found in an organism. An isolated material may be, but need not be, purified.

The term “purified” as used herein refers to material, such as a nucleic acid or polypeptide, that has been isolated under conditions that reduce or eliminate unrelated materials, i.e., contaminants. For example, a purified protein is preferably substantially free of other proteins or nucleic acids with which it is associated in a cell. As used herein, the term “substantially free” is used operationally, in the context of analytical testing of the material. Preferably, purified material substantially free of contaminants is at least 50% pure; more preferably, at least 90% pure, and more preferably still at least 99% pure. Purity can be evaluated by conventional means, e.g., chromatography, gel electrophoresis, immunoassay, composition analysis, biological assay, and other methods known in the art.

### Treatment of Lysosomal Storage Disorders

The method of the present invention provides a therapy for the treatment of lysosomal storage diseases, in particular, those lysosomal storage diseases which are not candidates for pharmacological chaperone therapy with small molecule inhibitors of the deficient lysosomal enzyme, because no such inhibitors have yet been identified and/or evaluated. Some examples of lysosomal enzymes falling into this category and their associated diseases can be found in Table 1, below. No therapies which directly address the underlying molecular defect exist for these diseases, and patients must rely on treatment of resulting symptoms which is often inadequate. Moreover, since many of these diseases have central nervous system involvement, enzyme replacement therapy is not a practical option since enzymes cannot cross the blood-brain barrier and would necessitate use of a catheter. However, the use of substrates as chaperones is not limited to lysosomal enzymes for which no small molecule chaperones have been identified (Table 2, below). To the contrary, this method is applicable to all enzymes.

The advantage of using substrates or even smaller substrate analogs or derivatives is that that will be able to cross the blood-brain barrier following systemic administration. For example, low molecular weight depolymerized heparin derivatives, especially tetra- and disaccharides, have been demonstrated to cross the blood brain barrier (using cultured astrocytes as a model; Leveugle et al., J. Neurochem. 1998; 70: 736-44), suggesting that low molecular weight derivatives of other proteoglycans or glycosaminoglycans will also be able to cross the blood brain barrier. In addition, analogs based on N-acetylglucosamine also were shown to cross the blood brain barrier (Kisilevsky et al., Am J Pathol. 2004; 164:2127-2137).

TABLE 1
Lysosomal Enzymes with no Established Small Molecule Inhibitors

| LYSOSOMAL | | | |
|---|---|---|---|
| ENZYME | SYNONYM | ACTIVITY | DISEASE |
| Iduronate-2-sulfatase | 2-sulfo-L-iduronate 2-sulfatase, chondroitinsulfatase, | Exosulfatase hydrolysis of the C2 | MPS II |
| | Hunter corrective factor, iduronate sulfatase, iduronate | sulfate ester bond from the non- | Hunter |
| | sulfate sulfatase, iduronate-2-sulfate sulfatase, iduronide-2- | reducing terminal of iduronic acid | |
| | sulfate sulfatase, idurono-2-sulfatase, L-iduronate 2-sulfate | residues on glucosaminoglycans | |
| | sulfatase, L-idurono sulfate sulfatase, sulfatase, L-idurono-, | heparan sulfate and dermatan sulfate | |
| | sulfo-L-iduronate sulfatase, sulfoiduronate sulfohydrolase | | |
| Heparan-N-sulfatase | Sulfamidase; heparan sulfamidase; sulphamate | Exosulfatase hydrolysis of sulfate | MPS IIIa |
| | sulphohydrolase; N-sulfoglucosaminide sulfamidase; | moiety (C2 sulfamate bond) attached | Sanfilippo Type A |
| | heparan sulfate sulfatase | to the amino group at the non-reducing | |
| | | terminal glucosamine residue of | |
| | | heparan sulfate | |
| α-glucosaminide N- | α-N-acetyl-glucosaminidase; acetyl CoA: α-glucosaminide | Catalyzes the transfer of the acetyl | MPS IIIc |
| acetyltransferase | N-acetyltransferase; | group from acetyl-CoA to terminal | Sanfilippo Type C |
| | | alpha-linked glucosamine residues of | |
| | | heparan sulfate | |
| N- | 6S | Exosulfatase de-O-sulfation of α- | MPS IIId |
| acetylglucosamine-6- | α-D-2-deoxy-2-N-acetyl-glucosamine-6-sulfate sulfatase; 2- | linked glucosamine 6-sulfate residues | Sanfilippo Type D |
| sulfate sulfatase | acetamido-2-deoxy-D-glucose 6-sulfate sulfatase; N- | from the non-reducing terminal of | |
| | acetylglucosamine-6-sulfatase; O,N-disulfate O- | heparan sulfate | |
| | sulfohydrolase; choindroitin sulfatase; N- | | |
| | acetylglucosamine-6-sulfatase | | |
| N- | N-acetylgalactosamine-6-sulfatase; galactose-6-sulfatase; | Catalyzes hydrolysis of the 6-sulfate | MPS IVb |
| acetylgalactosamine- | GALNS | groups of the N-acetyl-D- | Morquio disease A |
| 6-sulfate-sulfatase | | galactosamine 6-sulfate units of | |
| | | chondroitin sulfate and of the D- | |
| | | galactose 6-sulfate units of keratan | |
| | | sulfate | |
| Arylsulfatase A | Arylsulfate sulfohydrolase A; cerebroside-3-sulfate- | Catalyzes hydrolysis of galactose-3- | Metachromatic |
| | sulfatase; sulfatase | sulfate residues (sulfate ester | leukodystrophy |
| | | hydrolysis) in a number of lipids such | |
| | | as cerebroside 3-sulfate; | |
| | | Catalyzes hydrolysis of ascorbate 2- | |
| | | sulfate and many phenol sulfates | |
| Arylsulfatase B | Arylsulfate sulfohydrolase B; N-acetylgalactosamine-4- | Catalyzes hydrolysis of 4-sulfate | MPS VI |
| | sulfatase; choindroitinase; choindroitin sulfatase | groups from N-acetylgalactosamine 4- | Maroteaux-Lamy |
| | | sulfate moieties on the | |
| | | glycosaminoglycans, dermatan sulfate | |
| | | and chondroitin sulfate | |
| Acid ceramidase | N-acylsphingosine deacylase; glycosphingolipid ceramide | Catalyzes hydrolysis of the | Farber's disease |
| | deacylase; N-acylsphingosine amidohydrolase | sphingolipid ceramide into | lipogranulomatosis |
| | | sphingosine and free fatty acid | |
| N- | GlcNAc-PO₄ transferase; uridine | Catalyzes the transfer of a-N- | Mucolipidosis II |
| Acetylglucosamine- | diphosphoacetylglucosamine-glycoprotein, | acetylglucosamine 1-phosphate | 1-Cell disorder; and |
| 1-Phosphotransferase | acetylglucosamine-1-phosphotransferase, uridine | residues to high mannose | Mucolipidosis III |
| | diphosphoacetylglucosamine-lysosomal enzyme precursor, | oligosaccharide chains of lysosomal | Pseudo-Hurler |
| | lysosomal enzyme precursor acetylglucosamine-1- | enzymes, resulting in the formation of | Poldystrophy |
| | phosphotransferase, N-acetylglucosaminyl | a diester bond | |
| | phosphotransferase, N- | | |
| | acetylglucosaminylphosphotransferase, UDP- | | |
| | acetylglucosamine:lysosomal enzyme N- | | |
| | acetylglucosamine-1-phosphotransferase, UDP- | | |
| | GlcNAc:glycoprotein N-acetylglucosamine-1- | | |
| | phosphotransferase, UDP-GlcNAc:lysosomal enzyme N- | | |
| | acetylglucosamine-1-phosphotransferase, UDP-N- | | |
| | acetylglucosamine:glycoprotein N-acetylglucosamine-1- | | |
| | phosphotransferase, UDP-N- | | |
| | acetylglucosamine:glycoprotein N-acetylglucosaminyl-1- | | |
| | phosphotransferase, UDP-N-acetylglucosamine:lysosomal | | |
| | enzyme N-acetylglucosamine-1-phosphotransferase | | |

TABLE 2
Lysosomal Enzymes and Actual or Potential Small Molecule Chaperones

| | | SMALL MOLECULE |
|---|---|---|
| LYSOSOMAL ENZYME | DISEASE | CHAPERONE |
| α-galactosidase A | Fabry disease; Anderson-Fabry | 1-deoxygalactonojirimycin; a-allohomonojirimycin; a- |
| | disease | galactohomonojirimycin; b-1-C-butyl- |
| | | deoxynojirimycin; calystegines A3 and B2 and N- |
| | | methyl calystegines A3 and B2 |
| Acid β-glucosidase | Gaucher disease | Isofagomine; N-dodecyl-deoxynojirimycin; calysterines |
| | | A3, B1, B2 and C1 |
| Acid α-glucosidase | Pompe disease | 1-deoxynojirimycin; α-homonojirimycin; |
| | | castanospermine |
| α-L-iduronidase | Hurler-Scheie disease | 1-deoxyiduronojirimycin; 2-deoxy-3,4,5- |
| | | trideoxypiperidine |
| Iduronate sulfatase | Hunter disease | 2,5-anhydromannitol-6-sulphate; |
| | | suramin |
| β-galactosidase | G_{M1}-gangliosidosis; | 4-epi-isofagomine; 1-deoxygalactonojirimycin |
| | Morquio disease B | |
| β-glucuronidase | Sly disease (MPS VI) | 6-carboxy-isofagomine; 6-carboxy-3,4,5- |
| | | trihydroxy piperidine |
| α-fucosidase | Fucosidosis | 1-deoxyfuconojirirmycin; b-homofuconojirimycin; |
| | | 2,5-imino-1,2,5-trideoxy-L-glucitol; 2,5-dideoxy- |
| | | 2,5-imino-D-fucitol; 2,5-imino-1,2,5-trideoxy-D- |
| | | altritol; |
| Acid sphingomyelinase | Niemann-Pick A and B | desipramine; |
| | | phosphatidylinositol-4,5-diphosphate |
| β-hexosaminidase A | Tay Sachs disease | 2-N-acetemido-isofagomine; 1,2-dideoxy-2-acetamido- |
| | | nojirimycin; nagstatin |
| β-hexosaminidase B | Sandhoff disease | 2-N-acetemido-isofagomine; 1,2-dideoxy-2-acetamido- |
| | | nojirimycin; nagstatin |
| β-galactocerebrosidase | Krabbe disease | 2-N-acetamido-isofagomine |
| Acid ceramidase | Farber disease | N-oleoylethanolamine; |
| | | (1S,2R)-2-N-myristoylamino-1-phenyl-1-propanol; |
| | | (1R,2R)-2-N-myristoylamino-1-(4-nitrophenyl)- |
| | | 1,3-propandiol; |
| α-N-acetyl-glucosaminidase | Sanfilippo disease B (MPS IIIb) | 1,2-dideoxy-N-acetimido-nojirirmycin |
| acid α-mannosidase | α-mannosidosis | 1-deoxymannonojirimycin |
| acid β-mannosidase | β-mannosidosis | 2-hydroxy-isofagomine |
| α-N-acetylgalactosaminidase | Schindler-Kanzaki disease | 1,2-dideoxy-N-acetamido-galactonojirimycin |
| α-N-acetyl-neuraminidase | Sialidosis | 2,6-dideoxy-2,6-imino-sialic acid; siastatin B |
| Arylsulfatase A | Metachromatic Leukodystrophy | Sodium 2-hydroxy-5-nitro-α-toluenesulfonate |
| arylsulfatase B (N-acetyl-galactosamine- | Maroteaux Lamy disease (MPS VI) | leukotriene C4 |
| 4-sulfatase) | | phospho-nucleic acids |

### Substrates and Substrate Analogs or Derivatives

According to the present invention, the substrates that can be used as chaperones are either the natural or physiological substrates for the enzyme or are analogs or derivatives of the natural substrate which can be hydrolyzed by the target enzyme (in the case of lysosomal enzymes).

Lysosomal Enzyme Substrates. Exemplary lysosomal enzymes and their substrates and substrate analogs/derivatives are provided in Table 3, below. In one embodiment, the candidate substrate or analog or derivative will have an optimal catalytic activity at a pH which is lower than the pH in the endoplasmic reticulum (neutral), so that little cleavage of the substrate chaperone would occur in the ER or during translocation of the lysosomal enzyme to the lysosome. Once in the lysosome, where the pH is lower (about 4.8), the substrate chaperone would be hydrolyzed and the enzyme, which is likely to be more stable at a lower pH, will be able to bind and hydrolyse the natural substrates found in the lysosome. For example, for many of the substrate analogs/derivatives in Table 3, below, the optimum pH is between about 4 and 5, with lower Km and Kcat at pH above 5 (see cited publications to Hopwood; Beilicki and Freeman in Table 2, below).

As one example, Freeman and Hopwood (J Biol. Chem. 1986) describe substrate analogs for heparan-N-sulfatase (sulphamate sulphohydrolase). The substrates having acidic optimum pHs, i.e., optimal for the lysosome, are those with a C-6 sulfate ester on the GlcNS residue of disaccharide substrates (e.g., GlcNS6S-Ido; GlcNS6S-Ido2S; and GlcNS6S-IdOA; see Table 2 below for descriptions).

TABLE 3

| LYSOSOMAL | NATURAL | SUBSTRATE ANALOGS/ | |
|---|---|---|---|
| ENZYME | SUBSTRATE | DERIVATIVES | REFERENCE |
| Iduronate-2-sulfatase | Heparan sulfate | O-(α-L-idopyranosyluronic acid 2-sulfate)-(1-4)-(2,5-anhydro-D- | Hopwood, Carbohydr Res. |
| | Dermatan sulfate | mannitol-l-t 6-sulfate (IdA-MS); | 1979; 69: 203-16 |
| | | L-O-(α-iduronic acid 2-sulphate-(1-4)-D-O-2,5-anhydro- | Hopwood and Muller, |
| | | mannitol (IdoA2S-anM); | Carbohydr Res. 1983; |
| | | L-O-(α-iduronic acid 2-sulphate)-(1-4)-D-O-2,5-anhydro-- | 122: 227-39 |
| | | mannitol 6-sulphate (IdoA2S-anM6S); | Bielicki et al., Biochem J. |
| | | O-(α-L-idopyranosyluronic acid)-(1-3)-,5-anhydro-D-talitol 4- | 2990; 271: 75-86 |
| | | sulfate (IdoA-anT4S); | Dean, J Inherit Metab |
| | | O-(α-L-idopyranosyluronic acid 2-sulfate)-(1-3)-2,5-anhydro-D- | Disorders. 1983; 6: 108-11 |
| | | talitol 4-sulfate (IdoA2S-anT4S); | |
| | | L-O-(α-iduronic acid 2 sulphate)-D-O-(α-glucosamine 6- | |
| | | sulphate)-(1-4)-L-O-(α-iduronic acid 2-sulphate)-(1-4)-D-O- | |
| | | anhydromannitol 6-sulphate (IdoA2S-GlcNH6S-IdoA2S- | |
| | | anM6S); | |
| | | L-O-(α-iduronic acid 2 sulphate)-(1-4)-D-O-(α-2- | |
| | | sulphaminoglucosamine)-(1 leads to 4)-O-(β-D-glucuronic or α- | |
| | | L-iduronic acid)-(1-4) D-O(α-N-acetylglucosamine-(1 leads to | |
| | | 3)-D-O-gulonic acid (IdoA2S-GlcNS-UA-GlcNAc-GlcOA); | |
| | | O-(β-D-glucopyranosyluronic acid)-(1-3)-2,5-anhydro-D-talitol | |
| | | 4-sulfate (GlcA-anT4S); | |
| | | O-(β-D-glucopyranosyluronic acid)-(1-3)-2,5-anhydro-D-talitol | |
| | | 6-sulfate (GlcA-anT6S); and O-(α-L-idopyranosyluronic acid)- | |
| | | (1-3)-2,5-anhydro-D-talitol (IdoA-anT); | |
| | | O-(α-L-idopyranosyluronic acid-2-sulphate)-(1-4)-2,5-anhydro- | |
| | | D-mannitol-6-sulphate | |
| Heparan-N-sulfatase | Heparan sulfate | O-α-2-sulphaminoglucosamine)-(1-4) O-L-(α-iduronic-acid2- | Freeman and Hopwood, |
| | Heparin | sulphate)-(1-4)-O-D-(2,5)-anhydro-mannitol 6-sulphate | Biochem J. 1986; 234: 83-92 |
| | | (GlcNS-IdoA2S-anM6S); | |
| | | O-(α-2-sulphaminoglucosamine)-(1-4)-L-O-(α-iduronic acid)-(1- | |
| | | 4)-O-D-(α-2-sulphaminoglucosamine)-(1-3)-L-idonic acid | |
| | | (GlcNS-IdoA-GlcNS-IdOA); | |
| | | O-(α-2-sulphaminoglucosamine)-(1-4)-O-L-iduronic acid | |
| | | (GlcNS-IdOA); | |
| | | O-(α-2-sulphaminoglucosamine 6-sulphate)-(1-3)-L-idonic acid | |
| | | (GlcN6S-IdOA); | |
| | | O-(α-2-sulphaminoglucosamine 6 sulphate)-(1-3)-L-idonic acid | |
| | | (GlcNS6S-IdOA); | |
| | | O-(α-2-sulphaminoglucosamine)-(1-4)-l-idose) | |
| | | (GlcNS-Ido); | |
| | | O-(α-2-sulphaminoglucosamine 6-sulphate)-(1-4)-L-idose 2- | |
| | | sulphate (GlcNS6S-Ido2S); O-(α-2-sulphaminoglucosamine 6- | |
| | | sulphate)-(1-4)-L-idose (GlcNS6S-Ido); | |
| | | O-(α-2-sulphaminoglucosamine)-(1-4)-L-6-idose 2-sulphate | |
| | | (GlcNS-Ido2S); | |
| | | 2-sulphoamino-glucosamine (GlcNS); | |
| | | 2-sulphoamino-galactosamine (GalNS) | |
| α-glucosaminide N- | Heparan sulfate | α-N-acetylglucosamine; | Meikle et al., Biochem J. |
| acetyltransferase | | 0-(2-amino-2-deoxy-α-D-glucopyranosyl | 1995; 308: 327-333 |
| | | N-sulphate)-(1-4)-β-D-uronic acid-(1-4)-(2-amino-2-deoxy-α-D- | |
| | | glucopyranosyl N-sulphate)-(1-3)-L-idonicacid (or -2,5- | |
| | | anhydro-L-idonic acid or-L-gulonic acid); | |
| | | p-nitrophenyl α-D-mannoside; 4-methylumbelliferyl α-D- | |
| | | mannoside. | |
| N-acetyl-glucosamine- | Heparan sulfate | N-acetyl-glucosamine 6-sulfate; glucose 6-sulfate; | Kresse et al., PNAS. 1980; |
| 6-sulfate sulfatase | Keratan sulfate | O-α-D-6-sulfo-2-acetamido-2-deoxyglucosyl-(1-4)-O-uronosyl- | 77: 6822-26; |
| | | (1-4)-2,5-anhydro-D-mannitol (GlcNAc(6S)UA-aMan-ol); | Freeman and Hopwood, |
| | | O-(α-L-iduronic acid 2-sulphate)-(1-4)-D-O-(α-2- | Biochem J. 1987; 246: |
| | | sulphaminoglucosamine 6 sulphate)-(1-4)-L-O-(α-iduronic acid | 355-65; |
| | | 2-sulphate)-(1-4)-D-O-2,5-anhydro-mannitol 6-sulphate | Freeman and Hopwood, |
| | | (IdoA2S-GlcNS6S-IdoA2S-anM6S); | Biochem J. 1992; 282: |
| | | O-(α-N-acetylglucosamine 6-sulphate)-(1-4)-L-O-(α-iduronic | 605-14 |
| | | acid 2-sulphate)-(1-4)-D-O-2,5-anhydro-mannitol 6 | |
| | | sulphate(GlcNAc6S-IdoA2S-anM6S); | |
| | | O-α-glucosamine 6-sulphate)-(1-4)-L-O-(α-iduronic acid 2- | |
| | | sulphate)-(1-4)-D-O-2,5-anhydro-mannitol 6-sulphate | |
| | | (GlcNH6S-IdoA2S-anM6S); | |
| | | O-(α-N-acetylglucosamine 6-sulphate)-(1 leads to 3)-L-idonic | |
| | | acid (GlcNAc6S-IdOA) | |
| N-acetyl-galactosamine- | Keratan sulfate | Hyaluronidase-degraded C-6-S tetrasaccharide; | Singh et al., JCI. 1976; 57: |
| 6-sulfate-sulfatase | Chondroitin 6-sulfate | 6-sulfo-N-acetylgalactosamine- | 1036-1040; Pshezhetsky et |
| | | glucuronic acid-6-sulfo-N-acetyl-1-galactosaminitol; | al., JBC. 1996; 271: |
| | | N-acetylgalactosamine 6-sulfate-(β, 1-4)-glucuronic acid-(β, 1- | 28359-28365; |
| | | 3(_-N-acetyl-galactosaminitol 6-sulfate | Lim et al., Biochim |
| | | | Biophys Acta. |
| | | | 1981; 657(2): 344-55 |
| Arylsulfatase A | Cerebroside sulfate | 4-nitrocatechol sulfate; | Shapira and Nadler, Arch |
| | | dehydroepiandrosterone sulfate; | Biochem Biophys. |
| | | Cerebroside-3-sulfate; | 1975; 170(1): 179-87 |
| | | Ascorbate-2-sulfate; | Daniel and Chang, |
| | | Sodium 2-hydroxy-5-nitrobenzylsulfonate monohydrate (Na(+) × | Enzyme. 1990; 43: 212-22; |
| | | C(7)H(6)NO(6)S(−) × H(2)O); | Mehl et al., Biochim |
| | | N-[7-Nitrobenz-2-oxa-1,3-diazol-4-yl]psychosine sulfate (NBD- | Biophys Acta. |
| | | PS); | 1968; 151(3): 619-27; |
| | | 2-(1-pyrene)dodecanoyl cerebroside sulfate (P12-sulfatide); | lnoue et al, CMLS. 1986; |
| | | 12(1-pyrenesulfonylamido)dodecanoyl cerebroside sulfate | 42: 33-35; Chruszcz and |
| | | (PSA12-sulfatide) | Lewinski, Acta |
| | | | Crystallogr C. 2002; 58(Pt |
| | | | 3): m150-1; Louis et al., |
| | | | Mol Chem Neuropathol. |
| | | | 1991; 14(2): 113-30; |
| | | | Marchesini et al., Biochim |
| | | | Biophys Acta. 1989 |
| | | | 14; 1002(1): 14-9 |
| Arylsulfatase B | Iduronate sulfate | p-nitrocatechol sulfate; | Hwu et al., Zhonghua Min |
| | Dermatan sulfate | GalNAc4S-GlcA-GalitolNAc4S; | Guo Xiao Er Ke Yi Xue |
| | Chonidroitin sulfate | chondroitin 4-sulfate-tetrasaccharide; | Hui Za Zhi. |
| | | N-acetygalactosamine 4-sulfate-(1-4)-beta-glucuronic acid-(1-3)- | 1991; 32(5): 280-5; |
| | | beta-N-acetylgalactosaminitol 4-sulfate | Gibson et al., Biochem J. |
| | | | 1987; 248: 755-64; |
| | | | Gorham and Cantz, Hoppe |
| | | | Seylers Z Physiol Chem. |
| | | | 1978; 359(12): 1811-4. |
| Acid ceramidase | Ceramide | N-stearoylsphingosine; N-stearoyldihydro-sphingosine; N- | Momoi et al., Biochem J. |
| | | oleosphingosine; N-lauroylsphingosine | 1982; 205: 419-25 |
| N-Acetylglucosamine-1- | UDP-N- | α-methyl-mannoside | Ben-Yoseph et al., |
| Phosphotransferase | acetylglucosamine | | Biochem J. 1987; 278: |
| | | | 697-701 |
| α-galactosidase A | Ceramide trihexoside | α-D-galactosylamine; 4-methylumbelliferyl α-D- | Bishop and Desnick, J. |
| | | galactopyranoside; p-nitrophenyl α-D-galctopyranoside; | Biol. Chem 1981, 256: |
| | | | 1307-1316 |
| Acid β-glucosidase | Glucocerebroside | 2,3-di-O-tetradecyl-1-O-(beta-D-glucopyranosyl)-sn-glycerol; 4- | Glew et al., Biochem J. |
| (glucocerebrosidase) | | methylumbelliferyl β-D-glucopyranoside; p-nitrophenyl β- | 1991; 274(Pt 2): 557-563; |
| | | D-glucopyranoside; resorufin β-D-glucopyranoside | Schmuth et al., Journal of |
| | | | Investigative |
| | | | Dermatology. 2002; |
| | | | 119: 1298-1303; |
| Acid α-glucosidase | Glycogen | 4-methylumbelliferyl-α-D-glucopyranoside; p-nitrophenyl α-D- | |
| | | glucopyranoside; maltose | |
| α-L-iduronidase | Terminal desulfated α- | 4-methyhylumbelliferyl α-L-iduronide; α-L- | Dasgupta et al., |
| | 1-iduronic acid | idopyranosyluronic acid (1-3)-α, β-D-2-acetamido-2-deoxy- | Glycoconjugate J. 2004; |
| | residues of dermatan | 4-O-sulfo galactopyranose; O-(α-L-idopyranosyluronic | 17: 829-34; Hopwood et |
| | sulfate and of heparan | acid)-(1-3)-2,5 anhydro-D-talitol 4-sulfate (IdoA-anT4S); | al., Clin Genet. |
| | sulfate | 5-fluoro-α-L-idopyranosyluronic acid fluoride; (2-deoxy-2- | 1984; 26(5): 414-21; |
| | | fluoro-α-L-idopyranosyluronic acid fluoride (2FIdoAF); O- | Mrachko et al., |
| | | (α-L-idopyranosyluronic acid)-(1-4)-(2,5-anhydro-D- | Biochemistry 2003; 42: |
| | | mannitol-l-t 6-sulfate) (IdA--Ms); iduronosyl anhydro- | 8054-8065; Hopwood et |
| | | mannitol 6-sulphate | al., Carbohydr Res. |
| | | | 1979; 69: 203-16; |
| | | | Hopwood et al., Clin Sci |
| | | | (Lond). 1979; 57(3): 265-72 |
| β-galactosidase | G_{M1} gangliosides | O-β-D-galactopyranosyl-(1-4)-2,5-anhydro-D-mannitol 6-sulfate; | Hopwood et al., |
| | | O-β-D-galactopyranosyl-(1-4)-2,5-anhydro-D-mannitol 6-sulfate; | Carbohydr Res. |
| | | 6-octanoylamino-4-methylumbelliferyl β-D-galactopyranoside | 1983; 117: 263-74; Kaneski |
| | | and 6-butanoylamino-4-methylumbelliferyl β-D- | et al., Journal of Lipid |
| | | galactopyranoside; mono-, di-, and tri-sulfated β-Gal-β-GlcNac- | Research. 1994; 35: 1441-1451 |
| | | β-Gal-2,5-anhydro-D-mannitol; O-[4-(1-imidazolyl)butyl]-2,3- | |
| | | dicyano-1,4-hydroquinonyl β-D-galactopyranoside (Im-DCH- | |
| | | beta-Gal) and its tetraacetate derivative, Im-DCH-beta- | |
| | | Gal(OAc)4 | |
| β-glucuronidase | glycosaminoglycans | O-(β-D-glucopyranosyluronic acid)-(1-4)-(2,5-anhydro-D- | Marciniak et al., Clin |
| | | mannitol-l-t 6-sulfate); 4-nitrophenyl-β-D-glucuronide; 4- | Chem Lab Med. |
| | | methylumbelliferyl-β-D-glucuronide; O-(β-D- | 2006; 44(8): 933-7; Muller |
| | | glucopyranosyluronic acid)-(1-4)-2,5-anhydro-D-mannitol | and Hopwood; Clin. Chim. |
| | | | Acta. 1982; 123: 357-60 |
| α-L-fucosidase | Fucose-containing | 4-methylumbelliferyl-α-L-fucoside 2-Naphthyl α-L- | Gossrau, Histochemistry,. |
| | glycolipids | fucopyranoside; 2-chloro-4-nitrophenyl α-L- | 1977; 52: 259; Gu et al., |
| | | fucopyranoside; Fuc-α-(1-2)-galactose and Fuc-α-(1-2)- | Carbohydr Res. 2003 |
| | | galactose-β1-OC₆H₄NO_{2;} Fuc-α-(1-3)-GlcNac-β1-OC₆H₅; | 22; 338(15): 1603-7; |
| | | Fuc α-1-4 GlcNAc-β1-OC₆H₅ | DiCioccio et al., J. Biol. |
| | | | Chem. 1982; 257: 714-18. |
| Acid sphingomyelinase | Sphingomyelin | L-alpha-phosphatidyl-D-myo-inositol-3,5-bisphosphate | Kolzer et al., Biol Chem. |
| | | (PtdIns3,5P2); AD2765 (thiourea derivative of sphingomyelin); | 2003; 384(9): 1293-8; |
| | | 6-hexadecanoylamino-4-methylumbelliferyl-phosphorylcholine | Darroch et al., J Lipid Res. |
| | | | 2005 Nov; 46(11): 2315-24; |
| | | | Testai et al., J Neurochem. |
| | | | 2004; 89(3): 636-44 |
| Sialidase | Sialyloligosaccharides | 4-methylumbelliferyl-N-acetyl-α-D-neuraminic acid (Neu5Ac | Tiralongo et al., FEBS |
| | and glycopeptides | alpha 2MU); p-nitrophenyl-N-acetyl-α-D-neuraminic acid | Lett. 1995; 372(2-3): 148-50; |
| | | (Neu5Ac α-2PNP); 5-bromo-4-chloro-3-indoyl α-D-N-acetyl | U.S. Pat. No. 6,607,896; |
| | | neuraminic acid; α-S-(4-azido-2-nitrophenyl)-5-acetamido-2,6 | Warner, Biochem Biophys |
| | | anhydro-2,3,5,9-tetradeoxy-9-thio-D-glycero-D-galacto-non-2- | Res Commun. |
| | | enonic acid | 1987; 148(3): 1323-9 |
| β-hexosaminidase A | G_{M2}-gangliosides | naphthol-AS-Bl-N-acetyl-β-D-glucosaminide; 4-nitrophenyl-β- | Pennybacker et al., J Biol |
| | | acetyl-β-glucosamine; 4-methylumbelliferyl-2-acetamido-2- | Chem. |
| | | deoxy-β-D-glucopyranoside; p-nitrophenyl-2-acetamido-2- | 1996; 271(29): 17377-82. |
| | | deoxy-β-D-glucopyranoside | |
| β-hexosaminidase B | G_{M2}-gangliosides | 4-methylumbelliferyl-6-sulfo-2-acetamido-2-deoxy-β-D- | Pennybacker et al., J Biol |
| | | glucopyranoside | Chem. |
| | | | 1996; 271(29): 17377-82. |
| β-galactocerebrosidase | β-galactocerebroside; | 6-hexadecanoylamino-4-methylumbelliferyl beta-D- | Wiederschain et al., |
| | galactosylsphigosine | galactopyranoside; chromogenic 2-hexadecanoylamino-4- | Carbohydr Res. 1992 |
| | | nitrophenyl β-D-glucopyranoside | 7; 224: 255. |
| Acid ceramidase | ceramide | N-dodecanoylsphingosine; lauric acid; sphingosine | He et al., Anal Biochem. |
| | | | 1999; 274(2): 264-9; Okino |
| | | | et al., J Biol Chem. |
| | | | 2003; 278(32): 29948-53 |
| Acid α-mannosidase | α-linked mannose | p-nitrophenyl α-D-mannoside; 4-methylumbelliferyl α-D- | Khan et al., J. Biosci., |
| | residues from the non- | mannoside; 2(′),4(′)-Dinitrophenyl-α-D-mannopyranoside | 1982: 4(2): pp. 133-138; |
| | reducing end of N- | | Desmet et al., Anal |
| | linked glycoproteins | | Biochem. |
| | | | 2002; 307(2): 361-7 |
| Acid β-mannosidase | β-linked terminal | 4-methylumbelliferyl-beta-D-mannoside; Man-α-(1-3)[Manα(l- | McCabe et al., Enzyme. |
| | mannose residues | 6)]Manβ(1-4)GlcNAc (Man₃-GlcNAc₁); Man₃-GlcNAc₂ | 1990; 43(3): 137-45; Daher |
| | from N-linked | | et al., Biochem J. 1991; |
| | glycoproteins | | 277(Pt 3): 743-751 |
| α-N-acid β-mannosidase | Terminal non- | 4-methylumbelliferyl beta-N-acetylgalactosaminide; 4- | |
| acetylgalactosaminidase | reducing N-acetyl-D- | methylumbelliferyl-2-acetamido-2-deoxy-α-D- | |
| | galactosamine | galactopyranoside; p-nitrophenyl-2-acetamido-deoxy-D- | |
| | residues in N-acetyl-α- | galactopyranoside; aryl N-acetyl-α-D-galactosaminide | |
| | D-galactosaminides. | | |
| α-N- | heparan sulfate and | 4-methylumbelliferyl-2-acetamido-2-deoxy-α-D-glucopyranoside | Hopwood et al., Clin Chim |
| acetylglucosaminidase | heparin | (GlcNAc-IdOA); O-(α-2-acetamido-2-deoxy-D- | Acta. 1982; 120(1): 77-86. |
| | | glucopyranosyl)-(1-3)-L-idonic acid; O-(α-3-acetamido-2- | |
| | | deoxy-D-glucopyranosyl)-(1-4)-L-idose (GlcNAc-Ido); O-(α-2- | |
| | | acetamido-2-deoxy-D-glucopyranosyl)-(1-4)-1,6 anhydro-L- | |
| | | idose (GlcNAc-anIdo); O-(α-2-acetamido-2-deoxy-D- | |
| | | glucopyranosyl)-(1-4)-L-idose 2-sulfate (GlcNAc-Ido(OS); p- | |
| | | nitrophenyl-2-acetamido-deoxy-D-glucopyranoside. | |

Abbreviations: A dash between two numbers means “leads to” or “links to, e.g. (1-4) means “1 leads to 4.”

Iduronate-2-sulfatase. Bielicki et al. detailed the optimum pH and enzyme kinetics for iduronate-2-sulfatase for the substrate analogs listed in Table 2, above. The structure of the substrate affects the pH activity profile. Maximal activities towards the highly sulfated tetrasaccharide substrates IdoA2S-GlcNAc6S-IdoA2S-anM6S; IdoA2S-GlcNS6S-IdoA2S-anM6S; and IdoA2S-GlcNH6S-IdoA2S-anM6S were seen at 5.5, 5.7, and 5.1 respectively, although there was a pH range from about 4.6-6.5 for IdoA2S-GlcNAc6S-IdoA2S-anM6S, 5.0-6.5 for IdoA2S-GlcNS6S-IdoA2S-anM6S, and 4.2-6.0 for IdoA2S-GlcNH6S-IdoA2S-anM6S. At pH 6.3, the foregoing have about 80%, 90% and 12% respectively of their maximal activities.

The kinetics of the enzyme for the various substrate analogs at the optimum pH's for the substrate analogs as determined by Bielicki et al. are provided in Table 4, below. Briefly, the addition of a 6-sulfate ester group to the dissacharide IdoA2S-anM, resulting in IdoA2S-anM6S, results in a 63-fold increase in catalytic activity resulting from 5-fold and 13-fold increases, respectively, in binding affinity and turnover (Km and Kcat). The effect of the glucosamine substituent was to increase the binding affinity by up to 2-fold compared with GlcNAc and GlcNH.

TABLE 4

| | | | k_{cat.} | 10⁻⁶ × k_{cat.}/Kₘ | |
|---|---|---|---|---|---|
| | | | (turnover no.) | (catalytic | Relative |
| | pH | Kₘ | (mol/min per | efficiency) | catalytic |
| Substrate | optimum | (μM) | mol of enzyme) | (M⁻¹ · min⁻¹) | efficiency |
| IdoA2S-anM | 5.4 | 19.2 | 161 | 8.4 | 1.0 |
| IdoA2S-anM6S | 4.0 | 4.0 | 2114 | 529 | 63.0 |
| IdoA2S-anM6S | 5.0* | 2.5 | 905 | 362 | 43.1 |
| IdoA2S-anT4S | 4.0* | 1.1 | 270 | 246 | 29.3 |
| IdoA2S-anT4S | 5.0 | 0.7 | 507 | 724 | 86.2 |
| IdoA2S-GlcNS6S-IdoA2S-anM6S | 5.7 | 1.4 | 2177 | 1568 | 186.7 |
| IdoA2S-GlcNAc6S-IdoA2S-anM6S | 5.7 | 3.1 | 4858 | 1568 | 186.7 |
| IdoA2S-GlcNH6S-IdoA2S-anM6S | 5.4 | 2.5 | 1925 | 770 | 91.7 |
| IdoA2S-GlcNS-UA-GlcNAc-GlcOA | 5.4 | 1.9 | 756 | 399 | 47.5 |

*Not optimum pH

In summary, the aglycone structure adjacent to the non-reducing-end iduronate-2-sulfate residue influences the catalytic efficiency of the enzyme.

Lastly, sodium phosphate, sodium sulfate and sodium chloride salts are inhibitory for activity against the substrates whereas magnesium chloride, manganese chloride have no effect or increase the activity of the enzyme.

Heparan-N-sulfatase. As indicated above in Table 2, heparan analogs have been described by Freeman and Hopwood. This study also evaluated the pH optima of the purified enzyme for each of the substrate analogs. In general, the presence of C-6 sulfate ester on the GlcNS residue of the disaccharide substrates GlcNS-IdOA, GlcNS-Ido2S and GcINS-Ido (producing analogs GlcNS6S-IdOA, GlcNS6S-Ido2S and GlcNS6S-Ido, respectively), shifted the pH optimum from 5.5-6.7 to 3.8-4.2. By contrast, the addition of idose to GlcNS to produce GlcNS-Ido, increased the pH optimum from 5.6 to 6.7. The addition of a C-2 sulfate ester on the idose residue, to produce GlcNS-Ido2S and GlcNS-IdoA, lowered the pH optimum to 5.5. The pH optimum of the enzyme for the tetrasaccharide substrate GlcNS-IdoA-GlcNS-IdOA also was 5.5.

This study also evaluated the Km and Kcat of the substrates at the optimum pH ranges for heparan-N-sulfatase at 37°. These results are summarized in Table 5, below. In brief, the presence of C-6 sulfate esters on a substrate also containing a C-6 carboxy group on the adjacent monosaccharide residue (e.g., GlcNS6S-IdOA) increases the affinity for the enzyme (lowers the Km) but decreases the hydrolysis of the sulfamate bond (increases the Kcat). Thus, the presence of a C-6 sulfate ester on the non-reducing end of GlcNS residues would have a low Km and a low Kcat. In addition, since the pH optimum of the enzyme for GlcNS6S-IdOA is low (4.2), this substrate would be less likely to be hydrolyzed in the endoplasmic reticulum where the pH is neutral, freeing the enzyme to hydrolyze natural substrates e.g., in the acidic lysosome, where the pH is more optimal.

TABLE 5

| | | | k_{cat.} | | |
|---|---|---|---|---|---|
| | | | (turnover number) | k_{cat.}/Kₘ | Relative |
| | | | (mol/min per mol | (catalytic | catalytic |
| Substrate | pH | Kₘ (μM) | of enzyme) | efficiency) | efficiency* |
| GlcNS | 5.6† | 0.7† | 0.0021† | 3† | 1.00 |
| | | | (0.0005) | (0.73) | |
| GalNS | 5.6† | 16.1† | 0.0029† | 0.18† | 0.06 |
| | | | (0.0007) | (0.04) | |
| GlcNS-Ido | 6.7 | 7.7 | 0.0414 | 5.4 | 7.40 |
| GlcNS-Ido2S | 5.5 | 4.1 | 0.11 | 26.5 | 36 |
| GlcNS-IdOA | 5.6 | 35.0 | 9.19 | 262 | 359 |
| GlcNS-IdOA | 5.4† | 40.8† | 44† | 1078† | 1477 |
| GlcNS-IdoA-GlcNS-IdOA | 5.6 | 10.3 | 52 | 5057 | 6927 |
| GlcNS-IdoA2S-anM6S | 3.6 | 3.8 | 186 | 49051 | 67193 |
| | 5.6 | 5.3 | 117 | 22073 | 30237 |
| GlcNS6S-Ido | 4.2 | 3.0 | 0.061 | 20.2 | 28 |
| GlcNS6S-Ido2S | 3.8 | 4.1 | 3.333 | 283 | 388 |
| GlcNS6S-IdOA | 4.2 | 2.5 | 0.105 | 24.2 | 33 |
| | 5.6 | 2.4 | 0.088 | 36.7 | 50 |

ψ denotes 60° C.

### N-acetyl-glucosamine-6-sulfate sulfatase (6S).

As demonstrated by Freeman and Hopwood 1987, activity of 6S towards monosaccharides Glc6S and GlcNAc6S had a pH optimum of 5.7. Adding an α-(1-4)-idose residue or a β-(1-3)-galactitol residue results in a shift of the pH optimum from 5.7 to 5.4 or 5.0 respectively. The presence of a 6-carboxy group on the open-ring idose in GlcNAc6S-IdOA also shifts the pH optimum from 5.4 to 5.0.

The kinetic properties of 6S for various substrates is shown in Table 6, below. In brief, the simplest substrate was Glc6S, with a Km of 62.5 μM and a Kcat of 0.585 mol/min/mol enzyme. Addition of a 2-acetamido group to give GlcNAc6S result in a decrease in the Km by about 6-9-fold, and also a decrease in Kcat. Linking an idose to the GlcNAc6S to give GlcNAc6S-Ido has no effect on Km but decreases the Kcat. The addition of a 6-carboxy group to GlcNAc6S-Ido increases the turnover by about 80-fold. Substituents on the 2-amino group of the glucosamine 6-sulfate residue affect the activity of 6S on di- and trisaccharides. The un-substituted disaccharide and trisaccharide substrates have a lower turnover rate than the N-acetylated or N-sulfated equivalents.

TABLE 6

| | Form A | | | | | | | |
|---|---|---|---|---|---|---|---|---|
| | | | k_{cat.} | | | | | |
| | | | (turnover | | | | | Ratio of |
| | | | number) | 10⁻³ × | | | | catalytic |
| | | | (mol/min | k_{cat.}/Kₘ | Relative | Form B | | efficiencies: |
| | pH | Kₘ | per mol | (catalytic | catalytic | Kₘ | 10⁻³ × | form A/ |
| Substrate | optimum | (μM) | of enzyme) | efficiency) | efficiency* | (μM) | k_{cat.}/Kₘ | form B |
| Glc6S | 5.7 | 62.5 | 0.585 | 9.4 | 0.4 | 62.5 | 7 | 1.3 |
| GlcNAc6S | 5.7 | 7.1 | 0.165 | 23.2 | 1.0 | 10.0 | 11 | 2.1 |
| GlcNAc6S-Ido | 5.4 | 8.0 | 0.105 | 13.1 | 0.6 | 8.0 | 7.5 | 1.8 |
| GlcNS6S-Ido | 4.8 | 10.8 | 0.518 | 47.9 | 2.1 | 9.2 | 25.3 | 1.9 |
| GlcNAc6S-Ido2S | 4.5 | 3.6 | 0.165 | 45.8 | 2.0 | — | — | — |
| GlcNS6S-Ido2S | 4.2 | 2.3 | 0.201 | 87.5 | 3.8 | — | — | — |
| GlcNAc6S-IdOA | 5.0 | 11.1 | 7.90 | 712 | 31 | 14.3 | 299 | 2.4 |
| GlcNAc6S-IdOA | (5.0) | (10.0) | (6.79) | (679) | (29) | (13.2) | (241) | (2.8) |
| GlcNS6S-IdOA | 5.0 | 8.0 | 2.46 | 307 | 13 | 8.3 | 139 | 2.2 |
| GlcNS6S-IdOA | (5.0) | (7.6) | (1.725) | (227) | (9.8) | (6.6) | (91) | (2.5) |
| GlcNH6S-IdOA | 5.0 | 12.5 | 0.068 | 5.5 | 0.2 | 14.3 | 2.6 | 2.1 |
| GlcNS6S-IdoA2S-anM6S | 4.1 | 0.25 | 22.69 | 90760 | 3912 | — | — | — |
| GlcNAc6S-IdoA2S-anM6S | 4.3 | 0.76 | 7.08 | 9315 | 402 | — | — | — |
| GlcNH6S-IdoA2S-anM6S | 4.3 | 0.35 | 0.11 | 314 | 14 | — | — | — |
| GlcNAc6S-Galitol | 5.0 | 2.2 | 0.017 | 7.8 | 0.3 | 2.8 | 4 | 2.0 |
| GlcNAc6S-Gal-GlcNAc6S-Galitol | 4.5 | 1.7 | 0.042 | 25 | 1.1 | — | — | — |
| GlcNAc6S-Gal6S-GlcNAc6S-Galitol | 3.9 | 1.0 | 0.473 | 473 | 20 | — | — | — |

*k_{cat.}/Kₘ calculated relative to a value for GlcNAc6S = 1.

The conclusion from the foregoing studies is that the substrate analogs which possess structural features of the natural substrate generally result in the greatest rate of hydrolysis.

### Non-Lysosomal Storage Diseases

Increasing the degradation of proteoglycans, such as by increasing the activity of non-deficient lysosomal enzymes which degrade proteoglycans also is contemplated using substrates. For example, Alzheimer's disease is characterized by senile plaques composed of polymeric fibrils of beta amyloid (Aβ) 39-42-amino acid peptide formed after proteolytic processing of the amyloid precursor protein (APP). Heparan sulfate proteoglycans (perlecan) have been shown to colocalize with Aβ in Alzheimer's disease brain, and experimental evidence indicates that the interactions between the proteoglycan and the peptide are important for the promotion, deposition, and/or persistence of the senile plaques (Bame et al., J Biol. Chem. 1997; 272: 17005-11). Moreover, low concentrations of heparin recently were found to stimulate partially active BACEI, the enzyme that cleaves APP into Aβ peptide (Beckman et al., Biochemistry. 2006; 45(21):6703-14). Thus, one mechanism to prevent the formation of Aβ-heparan sulfate proteoglycan complexes that lead to deposition of amyloid would be to increase the degradation of heparan sulfate.

Since small molecule specific pharmacological chaperones have been shown to increase the wild-type as well as mutant lysosomal enzymes, there is reason to expect that substrate chaperones similarly will be able to stabilize wild-type lysosomal enzymes and increase their half-life and/or activity.

### Formulations, Administration and Dosage

The present invention provides that the substrates or analogs or derivatives of substrates can be administered in a dosage form that permits systemic administration, since it would be beneficial for the compounds to cross the blood-brain barrier to exert effects on neuronal cells. In one embodiment, the specific pharmacological chaperone is administered as monotherapy, preferably in an oral dosage form (described further below) with an appropriate pharmaceutically acceptable carrier, although other dosage forms are contemplated. Formulations, dosage, and routes of administration for the specific pharmacological chaperone are detailed below.

Formulations. Therapeutically effective substrates can be administered to an individual in standard formulations suitable for any route of administration. Standard formulations for all routes of administration are well known in the art. See e.g., Remington's Pharmaceutical Science, 20^{th} Edition, Mack Publishing Company (2000).

In one embodiment, the substrate or analog or derivative is formulated in a solid oral dosage form such as a tablet or capsule. The tablets or capsules can be prepared by conventional means with pharmaceutically acceptable excipients such as binding agents (e.g., pregelatinized maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets may be coated by methods well known in the art. Liquid preparations for oral administration may take the form of, for example, solutions, syrups or suspensions, or they may be presented as a dry product for constitution with water or another suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations may also contain buffer salts, flavoring, coloring and sweetening agents as appropriate.

In another embodiment, the substrate or analog or derivative is formulated for parenteral administration such as by continuous infusion or bolus injection. Formulations for injection can be aqueous or oily suspensions, solutions, dispersions, or emulsions depending on and may contain excipients such as suspending, stabilizing and/or dispersing agents. In all cases, the parenteral formulation must be sterile and must be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and polyethylene glycol, and the like), suitable mixtures thereof, and vegetable oils. The proper fluidity can be maintained, for example, by the use of a coating such as lecithin, by the maintenance of the required particle size in the case of dispersion and by the use of surfactants. Prevention of the action of microorganisms can be brought about by various antibacterial and antifungal agents, for example, parabens, chlorobutanol, phenol, benzyl alchohol, sorbic acid, and the like. Prolonged absorption of the injectable compositions can be brought about by the use in the compositions of agents delaying absorption, for example, aluminum monosterate and gelatin.

In a further embodiment, the substrate or analog or derivative can be delivered in a controlled-release formulation. Parenteral delivery systems for controlled release and include copolymer matrices such as polymers of lactic/glutamic acid (PLGA), osmotic pumps, implantable infusion systems, e.g., subcutaneous, encapsulated cell delivery, liposomal delivery, and transdermal patch.

Additional pharmaceutically acceptable excipients which may be included in the aforementioned formulations include buffers such as citrate buffer, phosphate buffer, acetate buffer, and bicarbonate buffer, amino acids, urea, alcohols, ascorbic acid, phospholipids; proteins, such as serum albumin, collagen, and gelatin; salts such as EDTA or EGTA, and sodium chloride; polyvinylpyrollidone; sugars, such as dextran, mannitol, sorbitol, and glycerol; propylene glycol and polyethylene glycol (e.g., PEG-4000, PEG-6000); glycerol; glycine or other amino acids; and lipids. Buffer systems for use with the formulations include citrate; acetate; bicarbonate; and phosphate buffers.

Administration. Exemplary routes of administration include oral or parenteral, including intravenous, subcutaneous, intra-arterial, intraperitoneal, ophthalmic, intramuscular, buccal, rectal, vaginal, intraorbital, intracerebral, intradermal, intracranial, intraspinal, intraventricular, intrathecal, intracisternal, intracapsular, intrapulmonary, intranasal, transmucosal, transdermal, or inhalation.

By way of example, heparan sulphate (HS) has been show to be orally active (Barsotti et al., Nephron. 1999; 81:310-316), as have other glycosaminoglycans (Baggio et al., Eur J Clin Pharmacol, 2004; 40: 247-40). Oral delivery of macromolecules, such as amphiphilic heparin derivatives, is described in U.S. Pat. No. 6,458,383 to Chen et al., and in U.S. Pat. No. 6,656,922 to Byun et al. Organic cation salts of sulfated glycosaminoglycans, including dermatan sulfate and heparan sulfate, which are suitable for oral or rectal administration, are described in U.S. Pat. No. 5,264,425. Formulations for oral delivery of agents, including heparan sulfate, are described in U.S. Pat. No. 6,761,903 to Chen et al. Moreover, low molecular weight depolymerized heparin derivatives, especially tetra- and disaccharides, have been demonstrated to cross the blood brain barrier (using cultured astrocytes as a model; Leveugle et al., J Neurochem. 1998; 70: 736-44), suggesting that low molecular weight derivatives of other glycosaminoglycans will also be able to cross the blood brain barrier.

The administered substrates or analogs or derivatives of the present invention can be targeted for cellular uptake using small peptides derived from human heparin binding proteins, which bind to extracellular heparan sulphate and are then endocytosed by lipid rafts (De Coupade et al., Biochem J. 2005; 390(Pt 2):407-18). It also has been demonstrated that exogenous hydrophobic molecules such as peptides can be taken up by cells and targeted to the ER (Day et al., Proc. Nall. Acad. Sci. USA. 1997; 94: 8064-8069; Patil et al., BMC Immunol. 2004; 5: 12), suggesting that oligosaccharide substrates could also be taken up. Drug modification can be used to increase delivery to the central nervous system. Such modifications include lipidization, structural modification to enhance stability, glycosylation, increasing affinity for nutrient transporters, prodrugs, vector-based, cationization, and polymer conjugation/encapsulation. See Witt et al., AAPS Journal. 2006; 8(1): E76-E88 for further description of these modifications. Specifically, Wan et al. describe uptake of chitosan oligosaccharide nanoparticles by A549 cells (Yao Xue Xue Bao. 2004; 39(3):227-31). Chitosan is a linear polysaccharide composed of randomly distributed β-(1-4)-linked D-glucosamine and N-acetyl-D-glucosamine.

In addition, it recently has been shown that translocation pores can transport small anionic molecules such as UDP-glucuronic acid into the ER (Lizak et al., Am J Physiol Cell Physiol. 2006; 291(3):C511-7) suggesting that the acidified forms of di-to tetrasaccharide substrates may be able to enter the ER via the same route (e.g., substrates containing iduronic acid).

Dosage. The dosage of the substrate or analog or derivative can be determined by routine experimentation. Pharmacokinetics and pharmacodynamic measures such as half-life (t_{1/2}), peak plasma concentration (Cmax), time to peak plasma concentration (tmax), exposure as measured by area under the curve (AUC), and tissue distribution will factor into selection of an appropriate substrate or analog or derivative, and an appropriate dosage of that substrate.

Data obtained from cell culture assay or animal studies may be used to formulate a therapeutic dosage range for use in humans and non-human animals. The dosage of compounds used in therapeutic methods of the present invention preferably lie within a range of circulating concentrations that includes the ED₅₀ concentration (effective for 50% of the tested population) but with little or no toxicity. The particular dosage used in any treatment may vary within this range, depending upon factors such as the particular dosage form employed, the route of administration utilized, the conditions of the individual (e.g., patient), and so forth.

The optimal concentrations of the substrate pharmacological chaperone are determined according to the amount required to stabilize and induce a proper conformation of the enzyme in vivo, in tissue or circulation, without preventing activity or bioavailability of the substrate in tissue or in circulation, or metabolism of the substrate chaperone in tissue or in circulation. In addition, off-target activity also should factor into any dosage determination so as to avoid any untoward or adverse side effects. For example, since heparan and dermatan sulfate are anti-coagulants, an analog or derivative lacking that property may be a better therapeutic candidate so as to prevent blood clotting in the event a subject bleeds. Since the degree of sulphation therefore appears to be an important functional property that contributes significantly to the anticoagulant effects of both heparan and dermatan sulfate, less sulfated analogs or derivatives, such as N-acetylated derivatives, may be better candidates for therapy (Ofosu et al., Biochem J. 1987; 248(3): 889-896; Patay et al., Biochem Soc Trans. 2005; 33(part 5): 1116-1118). Derivatives of heparin which exhibit diminished anti-coagulant activities are described in Lapierre et al., Glycobiology. 1996; 16: 366-66 and in U.S. Pat. No. 5,250,519.

### Assays and Screening

### Expression, Localization and Activity Assays

Evaluation of potential substrates or substrate analogs for chaperone activity can be achieved using routine assays. As indicated previously, enhanced expression of enzymes can be determined by measuring an increase in enzyme protein levels intracellularly, particularly in the ER, or by determining increased enzyme activity. Non-limiting exemplary methods for assessing enzyme activity are described below.

Determining intracellular expression. Methods for quantifying intracellular enzyme protein levels are known in the art. Such methods include Western blotting, immunoprecipitation followed by Western blotting (IP Western), or immunofluorescence using a tagged lysosomal protein.

Activity Assays. Activity assays of lysosomal proteins in the presence of a substrate are routine in the art. As one example, in vitro assays using purified lysosomal enzymes can be performed for use in determining kinetics for candidate substrates. Recombinant human sulfamidase can be prepared according to the method of Perkins et al., J Biol. Chem. 1999; 274: 37193-199. This method can be adapted for the preparation of other lysosomal enzymes. As another example, expression and characterization of human recombinant and alpha-N-acetylglucosaminidase and transfection into host cells is described in Weber et al., Protein Expr Purif. 2001; 21(2):251-9.

Means to assay enzyme activity and kinetics in the presence of fluorogenic (4-Methylumbelliferyl-α-D-N-sulphoglucosaminide) substrates is described in Karpova et al., J Inher Metab Dis. 1996; 19: 278-85. This method can be used on whole cell lysates to determine whether cells expressing mutant lysosomal enzymes and treated with a substrate have increased enzyme activity.

In one embodiment, use of differentially labeled substrates as chaperone and substrates for detection of activity is contemplated. For example, use of a substrate for chaperoning whose presence can be detected by absorbance, in combination with use of a substrate whose presence can be detected by fluorescence for determining activity.

Localization. Sensitive methods for visually detecting cellular localization also include fluorescent microscopy using fluorescent proteins or fluorescent antibodies. For example, enzyme proteins of interest can be tagged with e.g., green fluorescent protein (GFP), cyan fluorescent protein, yellow fluorescent protein, and red fluorescent protein, followed by multicolor and time-lapse microscopy and electron microscopy to study the fate of these proteins in fixed cells and in living cells. For a review of the use of fluorescent imaging in protein trafficking, see Watson et al., Adv Drug Deliv Rev 2005; 57(1):43-61. For a description of the use of confocal microscopy for intracellular co-localization of proteins, see Miyashita et al., Methods Mol. Biol. 2004; 261:399-410.

Fluorescence correlation spectroscopy (FCS) is an ultrasensitive and non-invasive detection method capable of single-molecule and real-time resolution (Vukojevic et al., Cell Mol Life Sci 2005; 62(5): 535-50). SPF((single-particle fluorescence imaging) uses the high sensitivity of fluorescence to visualize individual molecules that have been selectively labeled with small fluorescent particles (Chemy et al., Biochem Soc Trans 2003; 31(Pt 5): 1028-31). For a review of live cell imaging, see Hariguchi, Cell Struct Funct 2002; 27(5):333-4). Use of dual-fluorescent assays where both the target protein, e.g., lysosomal enzyme, and a lysosomal resident protein, e.g., lysosomal membrane protein-1 (LAMP-1), are differentially labeled, and then the two fluorescent signals overlaid, also can be used to confirm co-localization of the enzyme and the lysosomal resident protein in the lysosome. One specific assay using double-label immunofluorescence microscopy to determine the cellular location of heparan sulfatase is described in Muschol et al., Hum Mutat. 2004; 23(6):559-66.

Fluorescence resonance energy transfer (FRET) microscopy is also used to study the structure and localization of proteins under physiological conditions (Periasamy, J Biomed Opt 2001; 6(3): 287-91).

Animal models. Transgenic animal models such as mice expressing mutated lysosomal enzymes can be generated to assess enzyme activity and pharmacokinetics in vivo in response to treatment with substrates or analogs or derivatiaves. Methods of developing transgenic mice are well known in the art. For example, a transgenic mouse model expressing a mutant of N-acetylgalactosamine-6-sulfate sulfatase is described in Tomatsu et al., Hum Mol. Genet. 2005; 14(22):3321-35. Similar methods can be used to generate models of conformational mutant lysosomal enzymes.

### EXAMPLES

The present invention is further described by means of the examples, presented below. The use of such examples is illustrative only and in no way limits the scope and meaning of the invention or of any exemplified term. Likewise, the invention is not limited to any particular preferred embodiments described herein.

Indeed, many modifications and variations of the invention will be apparent to those skilled in the art upon reading this specification. The invention is therefore to be limited only by the terms of the appended claims along with the full scope of equivalents to which the claims are entitled.

### Example 1

### Use of Heparan Sulfate and Derivatives to Rescue Heparan-N-Sulfatase

### Methods

Transfections and/or cell culture. Stable or transient expression of conformationally mutant heparan-N-sulfatase into appropriate host cells (BHK, CHO, or COS-7) can be achieved using ordinary methods known in the art. Exemplary mutations of heparan sulfate are S66W, R150W, R206P and V486F. Alternatively, skin fibroblasts or another appropriate cell type (e.g., lymphocytes) from MPS11Ia patients can be cultured and used for evaluation (see Perkins et al., Mol Genet Metab. 2001; 73(4):306-12; Karpova et al., J Inherit Metab Dis. 1996; 19: 278-85).

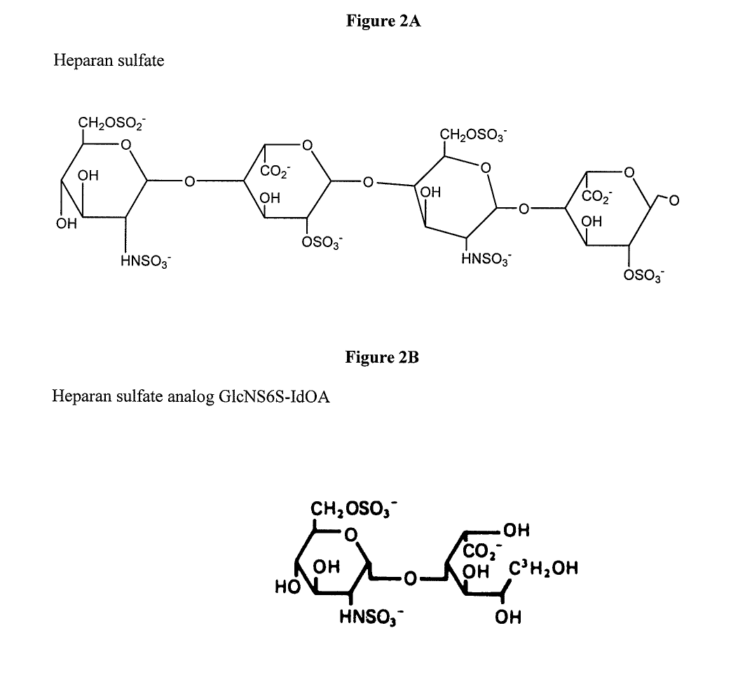

Substrate administration. Heparan (FIG. 2A) or analog GlcNS6S-IdOA (FIG. 2B) are added to cultures of the cells at varying concentrations (concentration response curve) and incubated under physiological conditions (37°, 5% CO₂) for a sufficient time. Substrates may be modified for improved uptake as described above (e.g., cationized).

Activity assay. Cells are then lysed and activity of heparan-N-sulfatase is measured in the lysates by the addition of a labeled substrate, such as 4-Methylumbelliferyl-α-D-sulfoglucosaminide (MU-αGlc-NS) according to the method of Karpova et al., J Inherit Metab Dis. 1996; 19: 278-85. Briefly, cell homogenates are prepared by ordinary means. The standard heparin sulphamidase reaction mixtures for fibroblasts and lymphocytes may consist of 10 μl homogenate (10 or 15 μg protein, respectively) and 20 μl MU-α-GlcNS (5 or 10 mmol/L, respectively) in Michaelis' barbital sodium acetate buffer, pH 6.5 (29 mmol/L sodium barbital, 29 mmot/L sodium acetate, 0.68% (w/v) NaCl, 0.02% (w/v) sodium azide; adjusted to pH 6.5 with HCl). The reaction mixtures are then incubated for 7 h at 37° C. The standard assay for leukocytes is as follows: 10 μl homogenate (60 μg protein) plus 20 μl mmol/L MU-αGlc-NS in barbital/sodium acetate buffer, pH 6.5 containing 0.225 mg/ml Pefabloc (a protease inhibitor). The cells are then incubated for 17 h at 47° C. For all assays, after the first incubation at either 37° C. or 47° C., 6 μl twice-concentrated Mctivain's phosphate/citrate buffer, pH 6.7, containing 0.02% sodium azide and 10 ul (0.1 U) yeast α-glucosidase (Sigma) in water is added and a second incubation of 24 h at 37° C. is carried out. Next, 200 μl 0.5 mob % Na2CO3/NaHCO r pH 10.7, was added and the fluorescence of the released 4-methylumbelliferone (MU) was measured on a fluorimeter and the fluorescence quantified.

Localization assays. Intracellular trafficking of cells harboring heparan-N-sulfatas can be achieved using double-immunofluorescence microscopy. For example, CHO cells can be grown and transfected with a vector containing wild-type or mutant heparan-N-sulfatase. Cells can be cultured for about 3 days, followed by treatment with 50 mg/ml cycloheximide in DMEM for 3 hr. Cells are then washed and fixed with methanol on ice for 5 min, washed again and blocked with PBS containing 1% BSA (PBSBSA).

Cells are then incubated using polyclonal rabbit anti-human sulfamidase:antibody (1:50) (see Muschol et al., Hum Mut. 2004; 23: 559-66) and either anti-LAMP1 antibody (1:15) or anti-PDI antibody (1:800) in PBS-BSA for 60 min at room temperature. Incubation with secondary antibodies is then performed at room temperature for 60 min using anti-mouse Cy3 (1:2,000) and anti-rabbit FITC (1:100) in PBS-BSA. Coverslips are mounted in fluorescent mounting medium and processed for immunofluorescence microscopy.

The double fluorescence was viewed with e.g., LSM 510 laser confocal microscope (Zeiss, Jena, Germany) set at excitation wave lengths of 488 (FITC) and 552 nm (Cy3), and emission wave lengths of 575 (FITC) and 570 nm (Cy3).

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

It is further to be understood that all values are approximate, and are provided for description.

Patents, patent applications, publications, product descriptions, and protocols are cited throughout this application, the disclosures of which are incorporated herein by reference in their entireties for all purposes.

1-28. (canceled)

29. A method of increasing the activity of a lysosomal enzyme in a cell, which method comprises contacting the cell with a substrate or substrate analog specific for the enzyme in an amount effective to increase the activity of the enzyme, with the proviso that the lysosomal enzyme is not acid sphingomyelinase.

30. The method of claim 29, wherein the lysosomal enzyme is selected from the group consisting of iduronate-2-sulfatase; heparan-N-sulfatase; α-glucosaminide N-acetyltransferase; N-acetyl-glucosamine-6-sulfate sulfatase; N-acetyl-galactosamine-6-sulfate-sulfatase; Arylsulfatase A; Arylsulfatase B; acid ceramidase; N-Acetylglucosamine-1-Phosphotransferase; α-galactosidase A; acid β-glucosidase; α-L-iduronidase; acid α-glucosidase; β-galactosidase; β-glucuronidase; α-L-fucosidase; sialidase; β-hexosaminidase A; β-hexosaminidase B; β-galactocerebrosidase; acid ceramidase; acid α-mannosidase; acid β-mannosidase; acid α-N-acid β-mannosidase acetylgalactosaminidase; α-N-acetylglucosaminidase; and β-N-acetylglucosaminidase.

31. The method of claim 30, wherein the lysosomal enzyme is α-galactosidase A and the substrate is selected from the group consisting of α-D-galactosylamine; 4-methylumbelliferyl α-D-galactopyranoside; and p-nitrophenyl α-D-galctopyranoside.

32. The method of claim 30, wherein the lysosomal enzyme is acid β-glucosidase and the substrate is selected from the group consisting of 2,3-di-O-tetradecyl-1-O-(beta-D-glucopyranosyl)-sn-glycerol; 4-methylumbelliferyl β-D-glucopyranoside; p-nitrophenyl β-D-glucopyranoside; and resorufin β-D-glucopyranoside.

33. The method of claim 30, wherein the lysosomal enzyme is acid α-glucosidase and the substrate is selected from the group consisting of 4-methylumbelliferyl α-D-glucopyranoside; p-nitrophenyl α-D-glucopyranoside.

34. The method of claim 30, wherein the lysosomal enzyme is β-galactosidase and the substrate is selected from the group consisting of O-β-D-galactopyranosyl-(1-4)-2,5-anhydro-D-mannitol 6-sulfate; O-β-D-galactopyranosyl-(1-4)-2,5-anhydro-D-mannitol 6-sulfate; 6-octanoylamino-4-methylumbelliferyl β-D-galactopyranoside and 6-butanoylamino-4-methylumbelliferyl β-D-galactopyranoside; mono-, di-, and tri-sulfated β-Gal-β-GlcNac-β-Gal-2,5-anhydro-D-mannitol; and O-[4-(1-imidazolyl)butyl]-2,3-dicyano-1,4-hydroquinonyl β-D-galactopyranoside (Im-DCH-beta-Gal) and its tetraacetate derivative, Im-DCH-beta-Gal(OAc)4.

35. The method of claim 30, wherein the lysosomal enzyme is heparan-N-sulfatase and the substrate or substrate analog is selected from the group consisting of heparan; heparin; O-α-2-sulphaminoglucosamine)-(1-4) O-L-(α-iduronic-acid 2-sulphate)-(1-4)-O-D-(2,5)-anhydro-mannitol 6-sulphate (GlcNS-IdoA2S-anM6S); O-(α-2-sulphaminoglucosamine)-(1-4)-L-O-(α-iduronic acid)-(1-4)-O-D-(α-2-sulphaminoglucosamine)-(1-3)-L-[6-³H]-idonic acid (GlcNS-IdoA-GlcNS-IdOA); O-(α-2-sulphaminoglucosamine)-(1-4)-O-L-iduronic acid (GlcNS-IdOA); O-(α-2-sulphaminoglucosamine 6-sulphate)-(1-3)-L-idonic acid (GlcN6S-IdOA); O-(α-2-sulphaminoglucosamine 6 sulphate)-(1-3)-L-idonic acid (GlcNS6S-IdOA); O-(α-2-sulphaminoglucosamine)-(1-4)-L-idose) (GlcNS-Ido); O-(α-2-sulphaminoglucosamine 6-sulphate)-(1-4)-L-[6-³H]-idose 2-sulphate (GlcNS6S-Ido2S); O-(α-2-sulphaminoglucosamine 6-sulphate)-(1-4)-L-idose (GlcNS6S-Ido); O-(1-2-sulphaminoglucosamine)-(1-4)-L-6-idose 2-sulphate (GlcNS-Ido2S); 2-sulphoamino-glucosamine (GlcNS); and 2-sulphoamino-galactosamine (GalNS).

36. The method of claim 30, wherein the lysosomal enzyme is α-glucosaminide N-acetyltransferase and the substrate or substrate analog is selected from the group consisting of heparan sulfate; α-N-acetylglucosamine; O-(2-amino-2-deoxy-α-D-glucopyranosyl N-sulphate)-(1-4)-β-D-uronic acid-(1-4)-(2-amino-2-deoxy-α-D-glucopyranosyl N-sulphate)-(1-3)-L-idonic acid or -2,5-anhydro-L-[6-³H]idonic acid or -L-gulonic acid).

37. The method of claim 30, wherein the lysosomal enzyme is N-acetyl-glucosamine-6-sulfate sulfatase and the substrate or substrate analog is selected from the group consisting of heparan sulfate; keratan sulfate; N-acetyl-glucosamine 6-sulfate; glucose 6-sulfate; O-α-D-6-sulfo-2-acetamido-2-deoxyglucosyl-(1-4)-O-uronosyl-(1-4)-2,5-anhydro-D-mannitol (GlcNAc(6S)UA-aMan-ol); O-(α-L-iduronic acid 2-sulphate)-(1-4)-D-β-(α-2-sulphaminoglucosamine 6 sulphate)-(1-4)-L-O-(α-iduronic acid 2-sulphate)-(1-4)-D-β-2,5-anhydro-mannitol 6-sulphate (IdoA2S-GlcNS6S-IdoA2S-anM6S);O-(α-N-acetylglucosamine 6-sulphate)-(1-4)-L-O-(α-iduronic acid 2-sulphate)-(1-4)-D-β-2,5-anhydro-mannitol 6 sulphate(GlcNAc6S-IdoA2S-anM6S); O-α-glucosamine 6-sulphate)-(1-4)-L-O-(α-iduronic acid 2-sulphate)-(1-4)-D-O-2,5-anhydro-mannitol 6-sulphate (GlcNH6S-IdoA2S-anM6S); and O-(α-N-acetylglucosamine 6-sulphate)-(1-3)-L-idonic acid (GlcNAc6S-IdOA).

38. The method of claim 30, wherein the lysosomal enzyme is β-glucuronidase and the substrate is selected from the group consisting of O-(β-D-glucopyranosyluronic acid)-(1-4)-(2,5-anhydro-D-mannitol-1-t 6-sulfate); 4-nitrophenyl-β-D-glucuronide; 4-methylumbelliferyl-β-D-glucuronide; and O-(β-D-glucopyranosyluronic acid)-(1-4)-2,5-anhydro-D-mannitol.

39. The method of claim 30, wherein the lysosomal enzyme is β-hexosaminidase A and the substrate is selected from the group consisting of 4-methylumbelliferyl-N-acetyl-α-D-neuraminic acid (Neu5Ac alpha 2MU); p-nitrophenyl-N-acetyl-α-D-neuraminic acid (Neu5Ac α-2PNP); 5-bromo-4-chloro-3-indoyl α-D-N-acetyl neuraminic acid; α-S-(4-azido-2-nitrophenyl)-5-acetamido-2,6 anhydro-2,3,5,9-tetradeoxy-9-thio-D-glycero-D-galacto-non-2-enonic acid.

40. The method of claim 30, wherein the lysosomal enzyme is β-hexosaminidase B and the substrate is selected from the group consisting of 4-methylumbelliferyl-6-sulfo-2-acetamido-2-deoxy-β-D-glucopyranoside.

41. The method of claim 30, wherein the lysosomal enzyme is β-galactocerebrosidase and the substrate is selected from the group consisting of 6-hexadecanoylamino-4-methylumbelliferyl beta-D-galactopyranoside; chromogenic 2-hexadecanoylamino-4-nitrophenyl β-D-glucopyranoside.

42. The method of claim 30, wherein the lysosomal enzyme is α-N-acetylglucosaminidase and the substrate is selected from the group consisting of 4-methylumbelliferyl-2-acetamido-2-deoxy-α-D-glucopyranoside (GlcNAc-IdOA); O-(α-2-acetamido-2-deoxy-D-glucopyranosyl)-(1-3)-L-idonic acid; O-(α-3-acetamido-2-deoxy-D-glucopyranosyl)-(1-4)-L-idose (GlcNAc-Ido); O-(α-2-acetamido-2-deoxy-D-glucopyranosyl)-(1-4)-1,6 anhydro-L-idose (GlcNAc-anIdo); O-(α-2-acetamido-2-deoxy-D-glucopyranosyl)-(1-4)-L-idose 2-sulfate (GlcNAc-Ido(OS); and p-nitrophenyl-2-acetamido-deoxy-D-glucopyranoside.

43. The method of claim 30, wherein the lysosomal enzyme is deficient due to a conformational mutation.
